SEQUENCE LISTING (1 sequence)

SEQ ID NO: 1 - DNA, 12698 bp, Artificial Sequence
  Description of Artificial Sequence Synthetic polynucleotide
aaacagtgtatccaggccaccaccgattgatcagggccagagaaacagacccagcagctgacctgccaccccgagccagaatactacagagtttttaagcccaaaatccacaatcatctgtgccaagttaccccaccagtcaggatttagggataggggacgtccttaggaacatgtctttgtggtacacctatcctgctcccattggttggggtattcagcagtggcaggggacttgcctagcattcatgtctcaacttgacagctaggatgtccgttaccaagggagctgctgggacttatactttatttgacccctatacaaagtggaatggcttttatttggttccttcaataataataataggaggaggatgagaagttcgaggatggctttgaacatattttgagttctaaaatagcctggactagatgtagccctgactccgaaaggacatggctccgtgggcgaaagggcttggcacacaagcctactgacctgatttcagtcccccagatgtagtcataataagtgatacatgagtaaaatttaaagcgaaaagtactagcaatgataataataaataaaacaaaataaaaaataaaacatgatttttttttctcctgaaaagatgtataggcattaggtttatatagttagttagttagttagttagttagttagttagttaagacaggtgtggttgtgtttagttaaggcaggtgtggttgtgtatgcctttaatctcggcactaaaagaggattaaaggagacaaggcaggtagatctctgagttcaagcctagccaggtttgcagagtgagttccaggacagccagggctacacagagaaaccctgtctcagaaaaccaaatagatggatagatagatagatagatagatagatagatagatagatagatagatagatagatagatagataaaatagttttaaaacatttatttacatttatttttatgtgtataactgttttgctttcatgtatgtctgtgtaccgtgtgtgtgcctggtgttagcagacagcagaactggaattaaagacagctatgagctgctatttgagttctgggaaccaaaccccagttccctgcaagaacggccagtgttgttaatctctaaaccatctcttccagccccatgcatttggtgtgtgtgtgtgtgtgtgtgtgtgtgtgtactattaattgcttttgagacagggtctcactatgtagctggccaggaacttgccacatagaacaggctgtcctcagactcatagagatctagctgcctctgtttcccatgtgctaagattaaagctgtgtgccaccatagcaagctggaaaggtcttatatacactttgaaaggatcaaaaagaacttgccagttcccagtttcaaaaactagaacgaatgtcctcggtgcgcttggcctccttaagaatggggggggggtattgttattgtctcttcacaagtgagaagagggacttgcccaaagtcacatgatgagagtgacagcattggacccagagcagctacttatacatcaaacacatccttgccctagaaataggtcttctagaaggcacccatgtgacttgcagagggtatctctatcttcgtggagacagggagccgggcttcctgttgctgtgtgcatcctgttgttctcttgttaggatgtccatcaaatgcatgcatttcctttcctaactctggacagtaacagtcgtctgcggccaagctgtgcctgaatgagcagaggcacaggcaccagccgtggccacccagcaaacatctctgctgactcagactggggtaaggcattccctaacagttagttaggtaggtaggtaggtaggtagtttgagacagagttttgctatgttgtctaagctggccttgaactcattatgttacccaacattaaacttacagcagccccagcctcagaatttactagattctgacacagagcagtctggcctcagcctcctgagtgctgggattacaggtatatgccaccatgcctggcaatccctaccagaacttttatttatttattttacttaaaaaaaaaacttttatttattttttaaatttttaaaaatttattttatttttatttacttactattagtgtgtgtgtggggggggaagatgtatatgtgggtgtgggtgcatgtatcagatatcgtgtgtcctgctctgctgatgtctaacgtactcctttgtaacaaggtctctggctgagcccagagctaggctggcagccagcaaactccagtgagcctctgtctgctccagacagtgctgaagttatgggtgtgtgtgtggccaggcccaacgttttatgtgagtgctgggaactaactcaggtccttaaggatgtcttatccactgagccatctctccagccaacacccttgcaacttgatttcttatattatagtgtcatgtggatgaaataaaattatgactgggaataatattaattgtatcagagttttatttacgtattttgtgtgcatgtgtgtatgtatgcatgtgtgtgtgcatgtcacagctggtttctgggagccagaggacaacttgctttgttggctctgtccctccatctttatttgggtccaggggttccaattcaggttgtctggcaggcacctttaccaactaatctgtctctccagccccaggagcagcagtctgcagaaattaaattccatactctcagttattgatgtagttgaggggcaggcagcatgtaaaactgctggggagctgctgcatgggggaggggggggctttggaaggctccactgggcagagaagcaaaggtaagtcccgaagggctatgaagccagaggccagatggcatgtttttccagaagcagcagatggccagctgggtggggcttgggggctagagcggccctgggtacacgcctttgtagcacagtgcactgctttgaaccctttatgtttaggcagcccagtgcactgcagtgggcagtgtactgagctgccccaaaaacacacaacagaagtcctacatgtcactgaaattttccctgtctacatagggatgtggaaatacacacacacacacacacacacacacacacacacacactcacacacatacacacacatgcattcacacacacacagagtctattctgagttaaaaaataaggaaaaatatgtcagtggtaaggcttttgtgaatgtgcaaggccttagatttgattccccagcaccacaaaaataaataaatacaaaacagagctgggcagtagtggtgcacacttgagaggcagaggcaggtggatgcctatagtttaaagccagtctgatctacatagagaggtcctgtctcaaataaataaaaaacgaacaaataagaaccaagaacaaaataaaatacattaaatgtgtatatcggtttatcagctcacagtgtctgacttcctctcttccttcctatgctgaatttcctcctgctaaaagataagtgaaaattttttaaaaggtctcttagaggacagttttatttggggggagttgattggtttgttgggtacagataaaactagcccttgctggctttgaactccatatagactgcaagttatttatttattcattgatttttgagacgaggtctccctaggccatcaggactgggcctgaactcagtcagtctgtaatccggacagggctcctgagtcgctgagacacagacctagtcaccaggtgtcctcctcttccttctatgtgggttaattttgaaataatacttattgcctttattaatcccagccgtgggaggcagaagccggtggatctctgagttcgaggccagcctggtttacaaagcaggctccaatacagccaggactacatagagaaattctgtcttggaggaaaataaaaaaaaaagctgggcagtcttggctcgtgcccttaatcccagcactagggagtttagggccagcctggtctacagaatgagtttcaggatagccagagatgcatgggaaaaccctgtctcagtaaaacaaaattatatttgtttatttatccattcatttattctcgtgtgtgtgtgtgtgtgtgtgtgtgtgtgtgtgtgtgtgtgtggtggtgtgttggggaagatgccatggtacgcgtgtggaagtaagaggacaatttgcattctaccacatgaagcccagggattaaactcagtacatcaagagtttttctcaagcggggggtggtggcgcacgcctttaatcccagcactggggaggcagaggcaggcagatttctgagttcaaggccagcctggtctacaaagtgagttccaggacagccagggctatacagagaaaccctgtctcgaaaaccaaaaaaaaaaaaaaaaaaaaaaaagtctttctctacctgtggagctatctcaatcacctctggcctttagttagttagttagttagtcaattagttaattttagagttttggggtagcattggctgtcctggaactagctctgtagaccaggctggccttgaactcacagagatccacctgcctctgcctccagagtgctgggatgagaggccatgtgccacccctctcggcctttcttttaattctttataaagtatgcccaagagttgcataagtctcttgcaaatgggagctataacccctccagcagacatctgagaactcttgcttgtttgttgatcactcccgggcttataaggtcaaatttcatgccccctagatagaatatttccacataacctgtttagaaaatttgtctttttagaaagatgtttaatttgctatcttgtgaagtttactaatgcttcatacaatattcacttgttctaagttttttcttccaaatccaggacatgcttatattaaaaaatgattctttatgtgatgtttaaataaatgggacttctatatcttatctagtacagttactttttaagacagagtttcacagaaccaaagatgactttgactcgacccccctgccttcaccttccaggtgctgtgcttgagctacgttccctacaggagtataaaaaaaggttgtgtttgaggcagggatttcctatgtagcccaggctggcctacaaactcacagagatctgtgtatgtcagcctgccgagtgctgggattaaaagcatgcaccaccactccataaaatatttatcttttgagtttatcttcccaacacttgaggtgaaaccaaagtattggctcccatccccaaacagttactaagcctttagtatcttaccaaagctggaagaacacagaaatcaaaatgagatataagccaggtttggtggcgcatacctgtctggaatcccagctgctttgaaacctgaagtaagagggtctacctgtgagaccatgtttcaaaaagcaaaagggaatctggccagcaagatggctttgagagtagaagtgagcagtaggaggcctgaaggcccgagtccaaatcccagaacccctggtagacctccacacacactctgtggcacactcatgcctgcaatacgcatcacatgtgagcacatatgtgtacacacacactaacaacaacaacaacaacaataacaataatagagaaaaacaatatccctacatcttcagttccacttctaggttttgatctcttggctggtggcccctaggaattcccctgggtttcaaagggaagtttcaggatttatctttttagaaagcaaaagaccaaaacatcttgcaagttacttctgcttgtttggaaacaaaaagacttagcttgaggaaaaaaggtaaatgcttggcagcaaactataaatggatatttttggggttggtgagatggctcagcagtcaagagctcttccgaaggtcctgagttcaaatcccagcaactacatgatggctcacaaaccatccataatgagatctgacgccctcttctagtgtgtctaaagacagctacaatgtacttacatatataattataaataaatctttgggctggagcaagtttgcatggcataacttcgtatagcatacattatacgaagttatctttcccctgggttcaaagggaagtttcaggatttatctttttagaaagcaaaagaccaaaacatcttgcaagttacttctgcttgtttggaaacaaaaagacttagcttgaggaaaaaaggtaaatgcttggcagcaaactataaatggatatttttggggtagagtgagcagaggccctgagttcaattcccagcaaccacataatggcttacaaccatctgtacagttacagtgtaatcatataaataaaataaataagtctttaaaattttttttgtgtgtgctggtgtagtgttggcacatgcctttaatcccagcacgtgggaagcagaggcaggcggatttctgagtttgaggccagcctggtctacagagtgaattccaggacagccagggctacagagagaaaccttgtcttaaaaaaccaaaaaaaattttaaaaattaaaaaataaatggatattttcggcacctgggtaaatgcttgattggtgtcagctctcagttgacgtcatcacctgttaacattcaaggattcttttctccttgcccacagcccagtcttctgggacagctccagtagaagaagccaaagcccatccatggagggatgccgggagacagaagtgaccaacggctccaacggtggcttggagttcaaccccatgaaggagtacatgatcctgagcagtggccagcagatcgccgtggcggtgctgtgcaccctgatggggctgctgagcgccctggagaacatggccgtgctctatattatcctgtcctcccggcggctccgcagaaagccctcgtacctgttcatcagcagcttggctggagctgacttcctggccagcgtgatcttcgcctgcaactttgtcatcttccacgtcttccacggggtcgactccaacgctatcttcctgctgaagatcggcagtgtgaccatgaccttcacagcctctgtgggcagcctgctgctaaccgctgttgaccgctacctatgtctgtgttacccgcctacctacaaagctctagtcacccgtgggagggcactggtggccctctgtgtcatgtgggtcctctcagcattgatttcttacctgccgctcatggggtggacttgttgccctagtccctgctctgagcttttcccactgatccctaacgactacctactgggctggcttctattcattgccatcctcttttccggcatcatctatacctatgggtatgtcctctggaaagcccaccggcatgtagccaccttggctgagcaccaggacaggcaggtgcctgggatagctcggatgcggctagacgtgaggttggccaagactctgggcctggtgctggctgtgctgctcatatgctggttccctgcactggctctcatgggccacagcctggtcaccacgctgagtgaccaggtcaaggaggccttcgccttctgttccatgctgtgccttgttaactctatggtcaatcctatcatttacgccctgcggagtggagagattcgctctgctgcccagcactgcctgataggctggaagaagtatctacagggcctcggacctgaggggaaagaagaaggcccaaggtcctcggttacagaaacagaggctgatgtgaaaaccacctaggagccaggatccagaactccaggctgctccaactgctgacaccacctgtctttctactggaaacagcccgagtcagaagtccgttcagttcactccctcgaagagagaggggttccagacccggattctttctgaagccagttctagggtaatggaaacagatccccctttttgctaaggaatgctggttggctggaaggtggcctggtcatgcctaccttttccagtctgtggagaggggcctgtaagaatggcaggctctgttcatatcaaatcaggaaagccttctgactccaaagacctagaggccagaggggcctccagagatgatactaaaggattcaggagatctgtgaagacaagggacctgttctcctggaatctagagtaccccagccgtggggccactgccctcaacttcatagatgtccatgtcttccatagtgtggattctagactgtcctgctgaagatagagagaccagctgcttctggttcctgcttcatcctctgtggctgtagagctatgtgaggctctaccttcaggtcacccttgcctcttgggggtcaagaattatgatgccctaaggaccaaggatgttcgcttacatccttcagacagcgtcaggagctgtcagctcagggtatcctgttgttgggaaagggccctcccgacatcctgataagccttgaaagccattatgaccttaggtccttaggggcgcagaggaagctgggttcactggaggtacaaagtcttgaaccaattgcgtacgttcgtgggattgtgtccgtgtcgcgaagttcctatactttctagagaataggaacttcgttcgaacataacttcgtatagcatacattatacgaagttatggtaccgtacgcggacgaccaacggacgtggacacgaagcttgccagggtaccgtacgcggacgaccaacggacgtggacacggatctgcccagagaactctattccttgtgacattttgctgtgtctcacacagcactatcccagtcttgcctagcttcagtacaaagcaagatattcacacagattctttgggtcctaggctgttcccagtgatgcttgactcagccccactggcctcttccacactctggtcctgaaggataagcagaaggagttgggagggaagacttcctgctgcaaaccaggcatcctaggtccacagccccacgctgtgtctctgggtgcctgcctttgcccttcagtaaaggttgcagtcccaacagatggaggttcaaggcgctgctggtggtaggaggggtaggtggcactggggaccacagacacagtgaaaggaaaactcttaaacatttaatacttttgcttttagaagatccggcccgcagacaggacttccccattgcccaaaggaactaacagggtggtgtccagtgcaaagaaaaatcagcctccctaataaagcaaggaggtccactcggctctacagcctctcctaagcttctgcaaggtgacagtttgttttctctgtgaaggtcaacggcgtctcattttattcttcccaagacaagttctgtgcatcaaaagctgctgcaaattctaatcgctgtttgggaggatcaggggaagaagtcgattcgcccacgcttagtgatttagactgtatttcaacatcaacttgggggaggaagagacagaaaagacacggtgtttccttattactcctcaaaggaggaagtgcttggttctgtcaacataagccgatctctccaaggtgcctgacagaggaggagggaatacagtttccacagctgtgaattcctgcaccagctttggtccacacttatgtggtcttgatcaacccccgccctttaccttgctttggtttccttctcggtaaattgggaatggtggggaaaacaatgctctccagggccattaaaacctctcattgctctaacatgctgtggttatctctgcaaccatggagacctttgtgccatatggaccatgtgacttctggctcccatacttcctgaagttatgggcacacaccatataccattgggcgtgtgctgactgtagaggaattgggtgactggccatatgagttttagagattttcccagggcaagagggttctcttggctctcaatttttctggtccctcatgcctcagtggtcccctgtgtgtgggtcaggcatgcttgtttagcggtgccagactgaagctccggccttccggtgtaccaagcaagtgttgtactcctgagtcaggttttcttcagttagatgctaactaagctctctgagagaccctcaaagtttttgaggtgtagctcagtcagtagagtgctgcatctgcaagagcaaatctctgggttcaaaggtcagtactacctaaaaccaggaatggcagtgccatcccctacctgtaatcccagcactcctgaggtacaagcaggaagatcaggaatgcaaagttactgtagctgtaagttcgaagccagcctgagctagttgagacccccttacctcaaaataaatttaagttacaattctaaattaaattaaaatgtctagggctggagagatggcttagtggttaagagcacagactgcttttccagaggtcctgagttcaattcccagcaaccacatggtggctcacaaccatctgtaatggaatctgatgccctcttctggtatgtttgaagagagcgatggtgtactcatatacataaaataagtaaataaatctttttttaaaaaaatgtttaagttcctcggagttcttgttaagttcaacggacaaaagatagactaaaataaccacgtctgcccttaatctttcagcagccatttctctccctccgcataaagctccttctaaccttgttatttctcaatctcttcggcaactctccaatctgctaaactccacctgctggctggaggtatagcacagagaacccgcggctcctaaatagtcactaaataaaacgacctggcttcagccttcccctgaagattggtgcaacaacgtggacttctcccagttttccccacaccttgggtctttctcacttgcggggaatccggcaattaaatccttaggcgagtctacggaatttaaattcaatagagcagaaccagcaacgccgccttaagtctttctcccttccaatgccagagggcgctccagagccgcaagcctgaccccaccccctctccttccaagccaatcgttactctgagaggcggggctactcgccggcgcttgcgtgttaagaggggggcggtgcgggcccggaagcaaggatgtaggagctgaacttgagtggtgggctgtggactctgggatgctgctgctgctgctgctgctgctagtggctgcggcccaggcagtggccctggctccgcgccgcttcactccggactggcagagcttggactcgcggccactgccgagctggttcgatgaggccaagttcggggtgttcgtgcactggggggtgttctcggtgcccgcctggggcagtgaatggttctggtggcactggcagggcgatcggatgccggcctaccagcgcttcatgacagaaaactacccgcccggcttcagctacgccgacttcgcaccgcagttcacagcgcgcttcttccacccggatcagtgggccgaactctttcaggctgccggggccaagtgagtgcgggacgggcgcgaggcgggagcaggcaggccatagccggcgctgcctgacctcggaaggtccaggaccctacgttctgcatctcggaggaaggacgctaaagacgagaatccagctgaggatcagttaaaaaaataaataaataaaataaaataaaataaaattaaggcgggaaagcactgggtggtacacctgtgcggagagagactagaatcaggctacatcggtgtctcgtttgaaaggtccagaacacagcccagattcccgtcggtggccctgagttgtagatctgaccagtccccttaggctacgggtagcgtccgtccgtggaagcttataaaaaagcgccgcctgacttccttgtagccctgtgcctctaaaggaagaaggaaggatggaagtttggggttgctctttgttggggtctccctcacttctcacttgcttatagtagaactcagaggaggaagggggttgtttgggcaaagccctgcagaggaagtgcgttttgagagccctctaagccagagaaaatattggctgactttggacgcgggatttggccactgtgagggcagcctcccgctcctggcttagtgtagtcctccgctctcttcattgattttctgccaaactccgactttttattccctcgggacgttaggttttctcccccgaagggaaaggctgtgtatagctggcacttagtattttcgccaagtaaaaggcctgagggaacagaaatctgtcatcttgccatcttgagagccgtaatttacaaagttgaactccagcctttttccagacttcctgtttaatggacgaggtaactagcccagagagaggaaggagaggaaggagcctggtgtcctagactgcaggtctatgatctggaagtgaaagctccagaaagctcatgggacaggtgtgcccaggctctgcaagccatacccagagggcgtggttatttgcttgttgagtgggaagggattttatatttcagaaggactttttgaaaggggaaagagatgtattgcaggaaggactgatgaagttcaggggcccctacaccagctagtggtaaaatctcatgtaacatgctatcttttcctataaaagtaagtgatatattgaatggactgattatctcaacacattttttttttcagtcctagatatttgagcctttgtcaccttaaatgacaggcagtttcacaatcttggttaagaaaacaagca

(19) United States
(12) Patent Application Publication
(10) Pub. No.: US 2019/0029238 A1
(43) Pub. Date: Jan. 31, 2019

(54) TRANSGENIC MICE

(71) Applicant: William Paterson University Of New Jersey, Wayne, NJ (US)

(72) Inventors: Emmanuel S. Onaivi, Bloomingdale, NJ (US); Qing-Rong Liu, Perry Hall, MD (US)

(21) Appl. No.: 15/552,698

(22) PCT Filed: Feb. 23, 2016
(86) PCT No.: PCT/US2016/019090

§ 371 Date: Aug. 22, 2017

Related U.S. Application Data
(60) Provisional application No. 62/121,227, filed on Feb. 26, 2015.

(51) Int. Cl. A01K 67/027; A61K 31/352; C07K 14/705; C12N 15/85

(52) U.S. Cl. CPC A01K 67/0276; A61K 31/352; C07K 14/70571; C12N 15/8509; A01K 2267/0356; A01K 2217/075; C12N 2015/8527; C12N 2800/30; A01K 2227/105

(57) ABSTRACT

The present invention provides a transgenic mouse and an animal model that is used to assay for the inhibition or activation of the Cnr2 gene and methods for screening drugs to treat or prevent psychosis, anxiety, depression, autism disorders, drug addiction, Parkinson's disease and/or Alzheimer's disease, multiple sclerosis, inflammation, stroke, osteoporosis, scleroderma or cancer.

### PRIORITY TO RELATED APPLICATIONS

This application claims the benefit of U.S. Provisional Application No. 62/121,227, filed Feb. 26, 2015. The entire contents of the above-identified application is hereby incorporated by reference.

### STATEMENT REGARDING FEDERALLY SPONSORED RESEARCH OR DEVELOPMENT

This invention was partially made with Government support under R15 DA032890-01A1. The Government has certain rights in the invention.

### BACKGROUND OF THE INVENTION

The present invention relates to a genetically modified mouse (transgenic mouse) wherein the mouse is able to produce a model of deletion of the Cnr2 gene in certain cell types. These cells include macrophages, monocytes, microglia, GABAergic, Glutamatergic, mono-aminergic cells in the periphery and neurons immune cells as well as brain glial cells.

There are three ways for transgenic mice to be produced. One way is the pronuclear injection of a gene into a single cell of the mouse embryo, where it will randomly integrate into the mouse genome. This method creates a transgenic mouse and is used to insert new genetic information into the mouse genome or to over-express endogenous genes.

The second way modifies embryonic stem cells with a DNA construct containing DNA sequences homologous to the target gene. Embryonic stem cells that recombine with the genomic DNA are selected for, and they are then injected into the mice blastocysts. This method is used to manipulate a single gene, in most cases “knocking out” the target gene. The disadvantages of these two germ line deletion methods include universal cell type gene deletions or interventions and developmental compensation.

The third way is site-specific recombination using Cre-Lox recombination technology that involves the targeting and splicing out of a specific gene with the help of a recombinase. Cre is expressed in a specific cell type, creating a cell-type specific deletion of the targeted gene. This method requires mating Cre mice and floxed (sandwich the targeted gene with loxP sequences) mice to produce conditional knockout mice with the targeted gene deleted in certain cell type.

The transgenic mice of the present invention are constructed in the third way. They are often called conditional Cre-Lox “knockout” mice because an activity of the gene is removed in a specific cell type. Such mouse models have been developed to study drug targets in a specific cell type related to obesity, heart disease, diabetes, arthritis, substance abuse, anxiety, aging and Parkinson's Disease.

Additionally, transgenic mice have been used to suppress genes to provide models for cancer therapies.

The Cnr2 gene that is the subject of the present invention encodes the cannabinoid receptor type 2 (CB₂). This is a G-protein coupled receptor and is related to the cannabinoid receptor type 1 CB₁. The CB₁ receptor is thought to be responsible for the pre-synaptic action of endocannabinoids, the psychoactive properties of tetrahydrocannabinol (THC) and other phytocannabinoids.

As stated, the Cnr2 gene encodes the CB₂ receptor which has 360 amino acids in humans. This G-protein coupled receptor has seven transmembrane spanning domains. They include a glycosylated extracellular N-terminus and an intracellular C-terminus.

There are two well characterized cannabinoid receptors (CBRs), CB1Rs and CB2Rs, with other candidates, such as GPR55, PPARs and vanilloid receptor (VP1, TRPV1) receptors that are thought to be involved with either the effects of cannabinoids and/or endocannabinoids (eCBs). Cannabinoids are the constituents in marijuana, and endocannabinoids (eCBs) are the endogenous marijuana-like substances found in animals and humans. The endocannabinoid system (ECS) consists of genes encoding cannabinoid receptors (CBRs), their endogenous ligands eCBs, and their enzymes involved in their syntheses and degradation of the eCBs (Ahn, K., M. K. McKinney and B. F. Cravatt (2008) (incorporated herein by reference) “Enzymatic pathways that regulate endocannabinoid signaling in the nervous system” Chem Rev 108(5): 1687-1701) CBRs are distributed in the brain and peripheral tissues. However, the neuronal and functional expression of CB₂Rs in the brain has been much less well studied and characterized in comparison to the expression of the ubiquitous CB₁Rs. Although earlier evidence suggested that CB₂Rs are present in the CNS, they were referred to as the peripheral CB₂Rs because many investigators were not able to detect neuronal CB₂Rs in healthy brains.

It has been found that functional neuronal CB₂Rs are expressed in brain and that activation and inhibition of these functional neuronal CB₂ cannabinoid receptors induce behavioral responses in motor function, cocaine addiction and emotionality tests in the rodent model.

Also CB₂Rs are associated with immune regulation and function, and as such, they are of interest to probe the role of CB₂Rs not only in neurological disorders associated with neuroinflamation but also in neuropsychiatric disturbances. Indeed studies have provided evidence for neuronal CNS effects of CB₂Rs and its possible role in drug addiction, eating disorders, psychosis, depression, and autism spectrum disorders.

The CNR2 cannabinoid gene (related to CB₂R) structure has not been well defined for the most part. However, many features of the CNR2 gene structure, regulation and variation are being defined with the use and identification of CB₂Rs in the mammalian CNS. This prior poor definition could be related to the previously held view that the CNR2 gene and CB₂Rs were not expressed in neurons in brain but mainly in immune cells. It was therefore less investigated for CNS roles except for the association with brain cells of macrophage lineage. The human CNR2 gene and its mouse and rat orthologs are located on chromosomes 1p36, 4QD3 and 5Q36, respectively. Genome-sequencing projects have also identified CNR2 genes in chimpanzee, dog, cow, chicken amphibian, puffer fish, and zebra fish. It appears that the human, rat, mouse and zebra fish genomes contain two isoforms of CB2Rs that have differential distribution patterns in the brain and peripheral tissues.

It has been discovered that the CNR2 genomic structure is species specific for expression patterns which account for differences between CNR2 genes in human and mice. With the discovery of a novel human CB₂R isoform, it has been discovered that the CB₂A isoform is predominantly expressed in human brain and testis and the promoter of CB₂A is located 45 kb upstream of the promoter of the previously identified CB2 gene (which is named CB2B isoform now), that is predominantly expressed in spleen. In contrast, CB2B mRNA expression could not be detected in brain regions in any significant level and is predominantly expressed in spleen. It has been found and reported that R63Q polymorphism in CNR2 gene is associated with alcoholism, depression, schizophrenia, and anorexia nervosa in Japanese subjects.

The distribution of the CB₂Rs has been resolved and some of the controversial issues associated with the detection and location of CB₂Rs in the CNS, by using CB2 isoform specific TaqMan probes that could differentiate the isoform-specific expression patterns and are more sensitive and specific than the CB₂ probes and primers previously used has been explained. It is thought that absence of CB₂R brain expression could be due to the low expression levels of CB₂A isoform in brain regions and the less specific CB₂R commercial antibodies in immunohistochemical studies, especially those studies using antibodies against human hCB2 epitopes for rodent brain immunostaining.

Further, unlike the present mice, there also are problems with the use of the CB₂ knockout (ko) mice that have been used in Western blots and in behavioral analysis. When the analyzed CB₂ knockout mice using the three TaqMan probes against two promoters of mouse CB₂ gene and the deleted part of CB₂ gene, are used, it is found that the promoters of CB₂R ko mice were still active and that a CB2 truncated version was expressed, indicating that the CB2 ko mice with ablation of the C-terminal peptides of 131 amino acids was an incomplete CB₂R knockout. Another mouse CB₂R ko mouse that has now been generated with ablation of the N-terminal peptide 156 amino acid may clarify the specificity of the antibodies that were used against the N-terminal epitopes. Unfortunately, this CB₂R-ko mouse is also an incomplete knockout as well. Nevertheless, many studies have now identified CB₂Rs in different brain regions, on neural progenitor cells of the subgranular zone of the dentate gyms in the hippocampus, and at CNS synapses in the entorhinal cortex (Morgan, N. H., I. M. Standord and G. L. Woodhall (2009) (incorporated herein by reference). “Functional CB2 type cannabinoid receptors at CNS synapses.” Neuropharmacology 57(4):356-368). Additionally, functional CB₂Rs are found in other neurons in the dorsal root ganglion, dopaminergic neurons in ventral tegmental area (VTA), and spinal cord, and activation of CB₂Rs on dorsal root ganglion-spinal cord neurons inhibit neuronal response to noxious stimuli, thereby contributing to the antinociceptive effects of CB2R agonists.

The CNS effects of CB₂Rs have been controversial and ambiguous (Liu, Q. R., C. H. Pan, A. Hishimoto, C. Y. Li, Z. X. Xi, A. Llorente-Berzal, M. P. Viveros, H. Ishiguro, T. Arinami, E. S. Onaivi and G. R. Uhl (2009). “Species differences in cannabinoid receptor 2 (CNR2 gene): identification of novel human and rodent CB2 isoforms, differential tissue expression and regulation by cannabinoid receptor ligands.” Genes Brain Behav 8(5):519-530) (incorporated herein by reference). Thus, the role in depression and substance abuse was unknown. The present invention provides a mouse model to advance understanding and using drugs in human subjects.

The involvement of brain neuronal CB₂Rs in drug abuse and depression is studied by using the conditional ko mice of the invention. Mice preferring alcohol have reduced Cnr2 gene expression in the ventral midbrain whereas the Cnr2 gene expression is unaltered in the ventral midbrain region of mice with little or no preference for alcohol. Treatment of mice with the putative CB₂R agonist JWH 015, enhances alcohol consumption in mice subjected to chronic mild stress (CMS), and the treatment with the CB₂R antagonist AM630, reduces the stress-induced increase in alcohol consumption. This CB₂R agonist or antagonist effect is absent in normal mice that were not subjected to CMS.

The expression of Cnr2 gene transcripts in rodents treated with opioids, cocaine and alcohol in comparison to control animals is useful. Animals treated with cocaine or heroin show increased Cnr2 gene transcripts in comparison to controls, indicating the presence of Cnr2 gene transcripts in the brain that is influenced by abused substances. Therefore, the pharmacological actions at brain CB₂Rs may be more complex than previously appreciated with species and sub-type differences and distribution patterns and are studied with the conditional ko mice of the invention.

The therapeutic potential of targeting CB₂Rs in brain has not been extensively characterized, perhaps in part due to its relatively low expression in brain or because of the lack of specific CB₂Rs and the long held believes that CB₂Rs were predominantly expressed in immune cells. Furthermore, the human CNR2 gene is about four times larger than that of rodents and some studies using antibodies against human hCB₂ epitopes for rodent brain immunostaining may have added to the CB₂ controversy and ambiguity (Liu, Pan et al., 2009). The present invention seeks to determine the specificity of a new CB₂R antibody designed using another CB₂R epitope “EHQDRQVPGIARMRLD” for use in studies. The specificity of this CB₂R antibody or other available specific antibody will undoubtedly resolve part of the controversy and ambiguity of CB₂Rs in the mammalian brain. The new knowledge from our data and those of other recent studies that CB₂Rs are present in the brain raises many questions about the possible roles that CB₂Rs may play in the nervous system.

In the present invention, the Cnr2-flox mouse line, when mated with for example, a gene promoter specific expressing Cre recombinase mouse line, is able to produce mouse models of complete deletion of Cnr2 gene in specific cell types, such as macrophage, monocytes, GABAergic, Glutamatergic, mono-aminergic systems in the periphery and in neurons and glial cells in brain.

The cell-type-specific deletion of Cnr2 gene provides a much desired animal model for developing pharmacological treatments for cancer, inflammation, neurodegeneration, osteoporosis and drug addiction, amongst other diseases.

The conditional Cnr2 mouse line with loxP flanking the full-length protein coding sequence is able to mate with a mouse line that expresses gene specific Cre recombinase, therefore producing a cell-type specific deletion of Cnr2. For instance, the offsprings of the floxed Cnr2 mice mating with Cx3crl Cre mice have Cnr2 deletion in macrophage in blood and microglia in brain. The conditional Cx3crl-Cnr2 knockout mouse model provides invaluable mouse models to develop effective treatment for chronic inflammation in peripheral and central systems that play causal roles in cancer and Alzheimer's disease. Another example is that the floxed Cnr2 mice mating with osteopontin (Opn) Cre mice to produce Cnr2 deletion in bone for development of treatment of osteoporosis is doable. In many other combinations including the conditional DAT-Cre-Cnr2-flox studies characterize and determine the role of CB2Rs in dopamine neurons. This is of huge importance in determining CB2R as a target in drug development for psychosis, anxiety, depression, autism disorders and drug addiction and neurological disturbances like Parkinson's and Alzheimer's disease.

The present invention is the first time that a floxed mouse line with site-specific loxP sites flanking Cnr2 fully protein coding exon and its 5′ splicing site has been created. The previous germ line knock out mouse lines are partial Cnr2 deletions of the C-terminal and N-terminal amino acid sequences, respectively. The germ line Cnr2 knock out mouse models have issues of developmental compensatory effects and lack cell or -tissue expression patterns that prevent the effective mouse models with cell type deletion of Cnr2 in order to study specific diseases such as cancer and Alzheimer's disease. The floxed Cnr2-Cre mice provide such models to investigate the inflammatory and molecular basis of CB₂ cannabinoid receptor function.

The present invention is exemplified with mouse models. Primate models may be more relevant to human diseases but are more expensive and gene targeted deletion of Cnr2 are more technically challenging. However, recent gene editing technology CRISPR-CAS9 successfully carried out in Rhesus monkey and that could be applied to Cnr2 gene locus in primate model. As such, other animal models are encompassed with the present invention.

Additionally, previous attempts to use CB₂ knock out mice have had certain issues. However, the use of the promoters of CB₂R mice show that these previous mouse models were unsuccessful, because the ablation of the C-terminal peptides of 131 amino acids resulted in an incomplete knockout.

The present invention overcomes these issues with a functional conditional knock out mouse that is a model for use in drug development and the development of mouse models for studying drug activities such as activation or inhibition of target cells.

### SUMMARY OF THE INVENTION

It is an object of the present invention to provide an animal model having deletions of the cannabinoid gene. It is a further object of the invention to provide an animal model comprising a floxed Cnr2 gene. The mouse is one of the animals useful as the animal model of the invention. The animal model of the invention has the Neo gene deleted from the Cnr2 gene and that gene is flanked with LoxP. More specifically, Seq ID No:1 is a gene sequence useful in the present invention. Another object of the invention is an animal model wherein said animal model comprises a Cre gene and LoxP genes flanking the CRB₂ gene coding region.

Cre genes selected for use in the present invention include, but are not limited to B6-Sjh-Slc6A3-creJ, (B6J.B6N(Cg)-Cx3crltml.1(Cre)Jung/J), B6(q-Tq(Nes0Cre)1.Kn₂ or B6.129-Oliq₂. Other mouse models are also useful for producing mice with the Cnr2-floxed mice. It is a further object of the invention to provide Opn-Cnr2 mice with osteocyte specific deletions of Cnr2 in order to have that mouse in an animal model to study osteoporosis It also is an object of the present invention to use the IL6-Cnr2 transgenic mice of the present invention with macrophage specific deletions of Cnr2. These provide models to study the effectiveness of such compounds as Ajulemic acid (Resunab®) for treating various immunological and/or autoimmune diseases such as but not limited to systemic sclerosis (scleroderma).

It is another object of the present invention to produce a mouse model wherein there is functional neuronal CB₂Rs induced behavioral responses in motor function and emotionality tests. These animals are mice and are named conditional knockout mice. They are used for drug screening in the BTBR T+tfJ mouse with autism behavioral phenotypes and up-regulated CB₂A gene expression in the brain. This is of significance with clinical implications to understanding the CNS effects of CB₂R acting drugs that have great potential therapeutic applications in pain, inflammation, auto-immune, mental and neurodegenerative disorders, drug and alcohol addiction.

It is a further object of the invention to have the Neo gene in a transgenic mouse deleted and have it flanked with LoxP. The sequence of SEQ ID NO:1 is useful for this model and for the transgenic mice of the present invention.

Additionally, the present invention uses the cassette found in FIG. 17. It is an object of the present invention to use the cassette identified in FIG. 17 to produce transgenic mice.

Another object of the present invention is to provide transgenic mice by crossing Cnr2-floxed mice with other mouse models, such as Cre gene related mice.

A further object of the invention is to provide a method for selecting a drug that targets the CB₂Rs. Examples of these methods include screening to discover medicines to treat drug addiction, Parkinson's Disease, post-stroke inflammation and to help reduce Central Nervous System (CNS) diseases such as Multiple Sclerosis (MS), Alzheimer's disease and other inflammations caused by neuronal injuries and/or ailments, such as cancers.

Another object of the present invention is to produce transgenic mice and mouse models for testing compounds that prophylactically and/or therapeutically are used to administer to patients with drug addiction ailments, alcohol addiction, neurological ailments such as Parkinson's Disease, Alzheimer's Disease, Multiple Sclerosis, Stroke, Post-Stroke Inflammation other Inflammation diseases, osteoporosis and cancer. This involves using the method of the invention to test or select drugs for prophylactically or therapeutically effects of these diseases.

These and further objects of the invention are illustrated, but not limited by the more detailed description of the invention provided herein below.

### BRIEF DESCRIPTION OF THE DRAWINGS

FIG. 1. Behavioral effects THC in a mouse model of depression: The time and number of immobility in the test is the index measured. The performance of the BTBR mice that exhibit autism-like phenotype in comparison to the control mice is shown. The data indicate that the BTBR mice are insensitive to the effects of THC compared to the control mice.

FIG. 2. Strategy of making Cnr2-floxed mice: (2A) Targeted iTL BA1 (129/SvEv×C57BL/6) hybrid embryonic stem cells are microinjected into C57BL/6 blastocysts. The resulting chimeras with a high percentage agouti coat color are mated to C57BL/6 FLP mice to remove the Neo cassette. Tail DNA is analyzed from pups with agouti or black coat color. Primer set NDEL1 and NDEL2 is used to screen mice for the deletion of the Neo cassette. (2B) The PCR product for the wild-type is 386 bp. After Neo deletion, one set of LoxP-FRT sites remain (˜159 bp). A second band with a size of 545 bp indicates Neo deletion. The presence of the Neo cassette is not amplified by this PCR screening because the size is too great. Triangles: LoxP sites; Rectangular: FRT sites for recombinase flipase deletion of drug selection marker Neo; LA (long arm), MA (middle arm), and SA (short arm): genomic regions for homologous recombination are done.

FIG. 3. Deletion of drug selection marker Neo: Primer set NDEL1 and NDEL2 is used to screen mice for the deletion of the Neo cassette. The PCR product for the wild-type is 386 bp (lower band). After Neo deletion, one set of LoxP-FRT sites remain (˜159 bp). A second band with a size of 545 bp (upper band) indicates Neo deletion (9579, 9582, 9560, 9564, 9566, and 9569).

FIG. 4. Presence of FLP (flipase) in Flp-mice: Primer set FLP1 and FLP2 is used to screen mice for the presence of the FLP transgene in Neo-deleted mice. The amplified product for primer set FLP1 and FLP2 is 725 bp.

FIG. 5. Screening for Distal LoxP Site: A PCR was performed to detect the presence of the distal LoxP site flaking coding exon using the SC1 and SDL2 primers. This reaction amplifies a wild type product 350 bp in size. The presence of a second PCR product 44 bp greater than the wild type product indicates a positive LoxP PCR in Neo-deleted mice.

FIG. 6. Confirmation of Short Homology Arm Integration: Tail DNA samples from positive mice are amplified with primers NEO-GT and A1. NEOGT is located inside the Neo cassette and A1 is located downstream of the short homology arm, outside the region used to create the targeting construct. NEO-GT/A1 amplifies a fragment of 4.34 kb in length. Due to the presence of the Neo cassette in the expanded ES cell, the amplified size is 6.31 kb.

FIG. 7. Absence of FLP Transgene: Primer set FLP1 and FLP2 is used to screen mice for the absence of the FLP transgene. The amplified product for primer set FLP1 and FLP2 is 725bp. (Mice C2274 and C2278 are FLP present and are sacrificed.)

FIG. 8A. Production and screening for homozygous Neo Deletion with LoxP flanking entire Cnr2 coding region: Primer set NDEL1 and NDEL2 is used to screen mice for the deletion of the Neo cassette. The PCR product for the wild-type is 386 bp. After Neo deletion, one set of LoxP-FRT sites remains (159 bp). A second band with a size of 545 bp indicates Neo deletion. A single band of 386 bp indicates a wild type mouse, two bands 386 and 545 bp in size indicates a heterozygous mouse, and a single band 545 bp in length indicates a mutant mouse. (C2626, C2627, and C2632 are homozygous Cnr2-floxed mice).

FIG. 8B. Further production and screening for homozygous Neo Deletion with LoxP flanking entire Cnr2 coding region: A single band of 386 bp indicates a wild type mouse, two bands 386 and 545 bp in size indicates a heterozygous mouse, and a single band 545 bp in length indicates a mutant mouse. (C2643, C2645, and C2648 are homozygous Cnr2-floxed mice).

FIG. 8C. Further production and screening for homozygous Neo Deletion with LoxP flanking entire Cnr2 coding region: A single band of 386 bp indicates a wild type mouse, two bands 386 and 545 bp in size indicates a heterozygous mouse, and a single band 545 bp in length indicates a mutant mouse. (C2671 is homozygous Cnr2-floxed mice).

FIG. 9. DNA listing of mouse #C2283. The sequence shaded is the Neo cassette. The underlined sequence is FRT, and the loxP site is red shaded. This provides the comparison of the DNA sequence of the invention to that of known DNA.

FIG. 10. Cnr2-floxed (CB2^{f/f}) mouse model. Homozygous Cnr2 transgenic mice with loxP flanking the entire coding region of exon 3 of CB2 cannabinoid receptor are produced. This is the first time Cnr2-floxed mice are available to generate cell type specific knockout CB₂R. The mice are thriving and reproducing for studying macrophage, microglia, and neuron specific (e.g. dopaminergic neuron) CB₂R effects. Those cell type specific CB₂R knockout mice are invaluable animal models for studying and development of effective therapy for cancer, pain, addition, neurodegenerative, autism and psychiatric disease.

FIG. 11. Provides Dat-Cnr2 mouse double allele genotyping Cnr2-flox mice: mutant allele is 545 bp; wild type allele is 386 bp. Dat-Cre mutant allele is 152 bp, wild type allele 264 bp. Homozygous double allele mutant Dat-Cnr2 mice are identified by genotyping #8-7.

FIG. 12. Provides Cx3crl-Cnr2 mouse double allele genotyping: Cnr2-flox mutant allele 545 bp; wild type allele 386. Cx3crl-Cre mutant allele 380 bp; wild type allele 819 bp. Homozygous Cnr2-flox mutant allele and heterozygous Cx3crl-cre mutant alleles of Cx3crl-Cnr2 mice are identified by genotyping #4-1.

FIG. 13. CB₂-O2 probe is used (506-934 bp of NM_009924.4; catalog No: 436091, Advanced Cell Diagnostics.) to hybridize deleted region of Cnr2 protein coding sequence.

FIG. 14. RNAscope in situ hybridization (ISH) of the ventral tegmental area (VTA) with Cnr2 and tyrosine hydroxylase (TH, DA neuron marker) probes. The CB2 mRNA is detected in most dopamine neurons of (A) wildtype (+/+; +/+) and (B) a few of heterozygous (−/−; −/+); while (C) absent in Dat-Cnr2 (−/−; −/−). White arrow heads represent DA neurons with CB2 mRNA, brown arrow heads DA neurons without CB2 mRNA, and green arrow heads non-DA neurons with CB2 mRNA.

FIG. 15. The performance of the DAT-Cnr2 in the plus-maze test of anxiety behavior, is evaluated and its found that Dat-Cnr2 homozygous mice are less anxious than heterozygous and wild type mice (FIG. 7, n=4-6). Performance in the elevated plus-maze test measuring time seconds and entry numbers into open and closed arms. This emotionality test is a measure of aversive behavior indicative of an anxiolytic index in the mouse model.

FIG. 16. Comparison of naive treated mice of Dat-Cnr2 homozygous mice with heterozygous and wild type mice other genotypes on cocaine stimulated wheel running activity is shown. Homozygous mice of Cnr2 deletion have higher locomotor activity than heterozygous and wild type mice on cocaine stimulation.

FIG. 17. This figure provides a detailed illustration of the cassette used in the invention and Cnr2-flox gene locus after homologous recombination and deletion of the Neo gene.

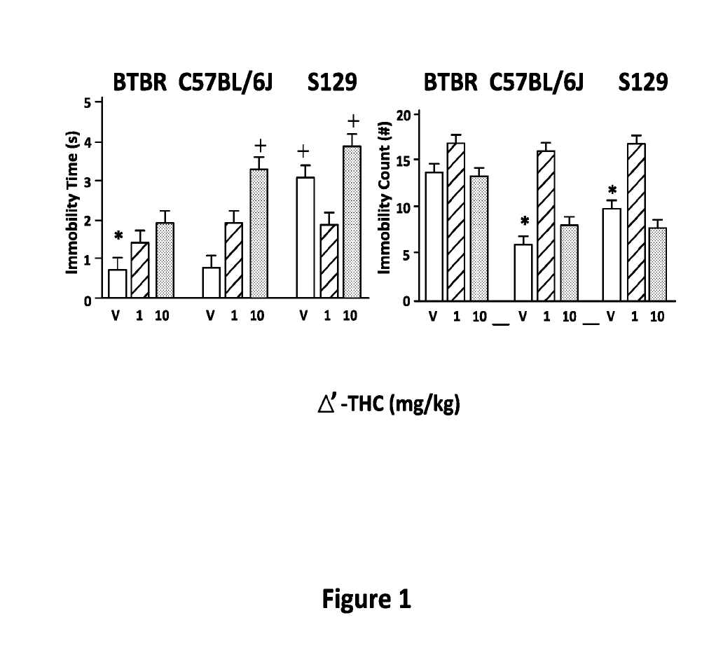

(Upper panel) Before homologous recombination and selection: 5′-arm includes Cnr2 exon2 and 3′-arm includes partial exon3 of 3′-UTR (un-translated region) for homologous recombination. loxP_site2 represents distal loxP sequence and loxP proximal loxP sequence for cell type specific deletion of Cnr2 protein coding sequence (5pr_exon3) and splicing acceptor site (5pr_Flank_Acceptor). Targeted region represents Cnr2 entire protein coding sequence and the splicing acceptor site sequence. Stop_seq represents stop codon. FRT_Neo_FRT_loxP represents Neo construct including FRT sequence, Neo flanking sequence and Neo gene. NDEL1_CB2F and NDEL2_CB2R represent genotyping primers for detection of Neo deletion after flipase recombination. The Neo gene is inserted in the exon3 that is interrupted into 5pr-exon3 and 3pr-exon3. (Lower panel) After homologous recombination and selection: Neo gene and the most of Neo flanking sequence are deleted by flipase recombination. The entire Cnr2 protein coding region and exon3 splicing site sequence are sandwiched by loxP sequence for the purpose of cell type specific deletion of complete CB2R protein.

### DETAILED DESCRIPTION OF THE INVENTION

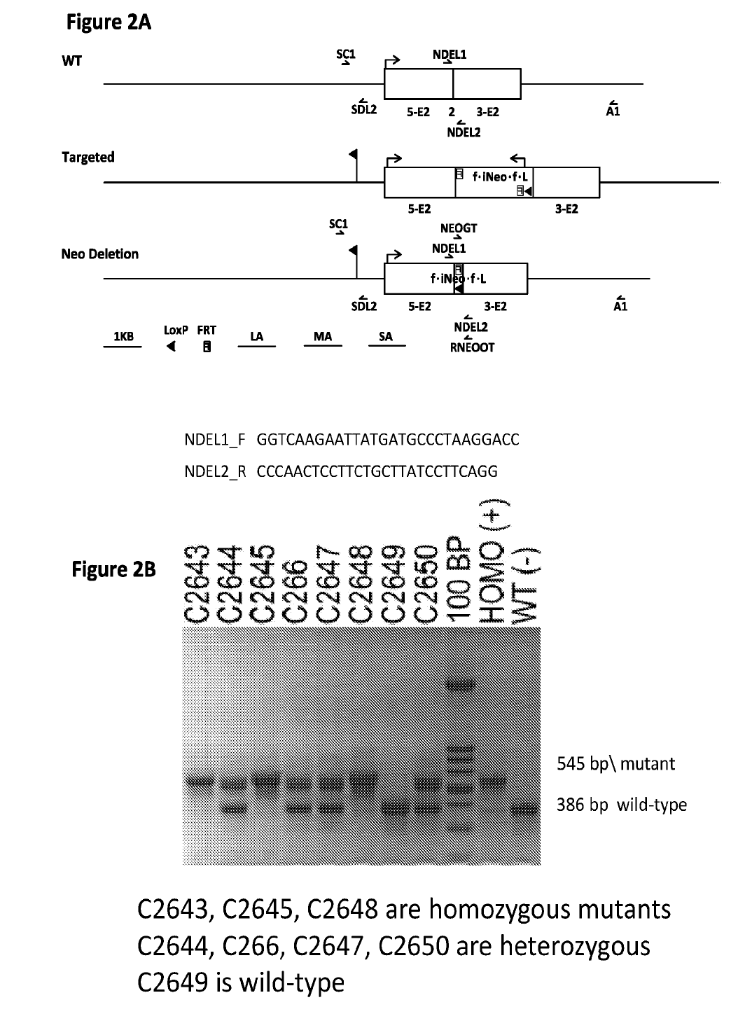

The present invention provides a floxed CB₂ receptor gene that has had the Neo cassette deleted. (See FIG. 2) Once this is accomplished, mice that had this gene are screened to ensure that the Neo-deleted gene is true. Screening is accomplished by utilizing F=LP (flipase) procedures, as well as other procedures known to those of ordinary skill in this area.

PCR is an effective procedure to test for the coding regions of the desired gene. However, other amplification methods such as but not limited to LAMP (loop-mediated isothermal amplification) also are useful for the present invention's use. This is inclusive of standard LAMP and rapid LAMP. Another amplification technique is Strand Displacement Amplification that is useful in amplifying the requested gene of the invention.

Primers are used to screen mice produced with the Neo cassette deletion of the floxed Cnr2 gene. These primers are the FLP1 and FLP2 primers to identify mice that do not have a FLP transgene.

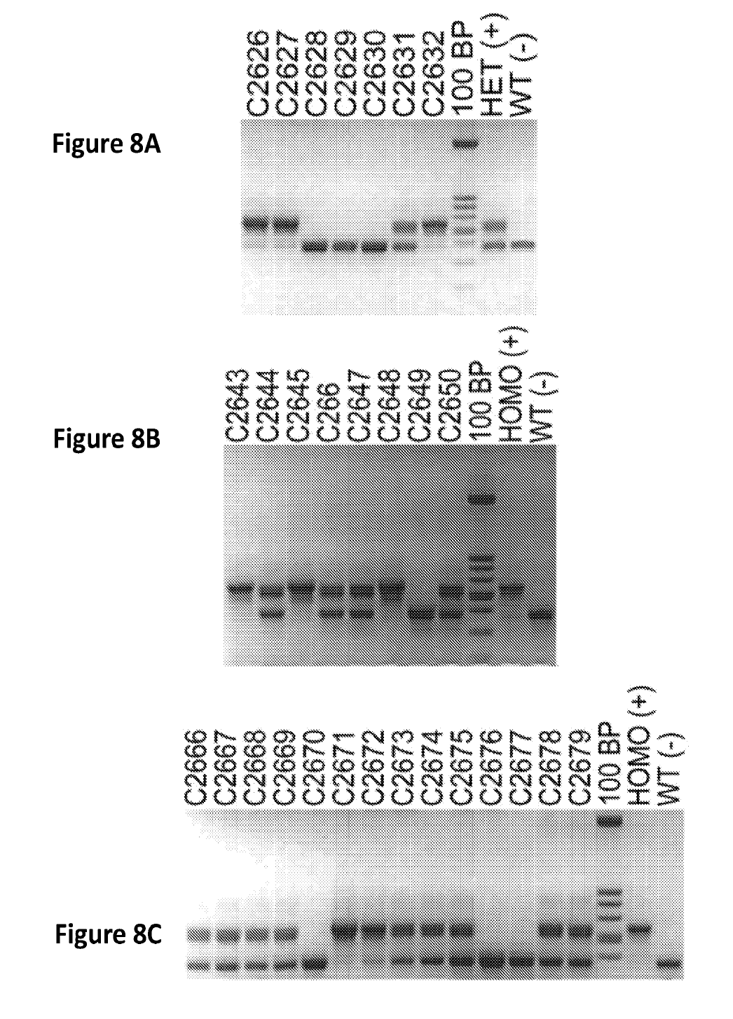

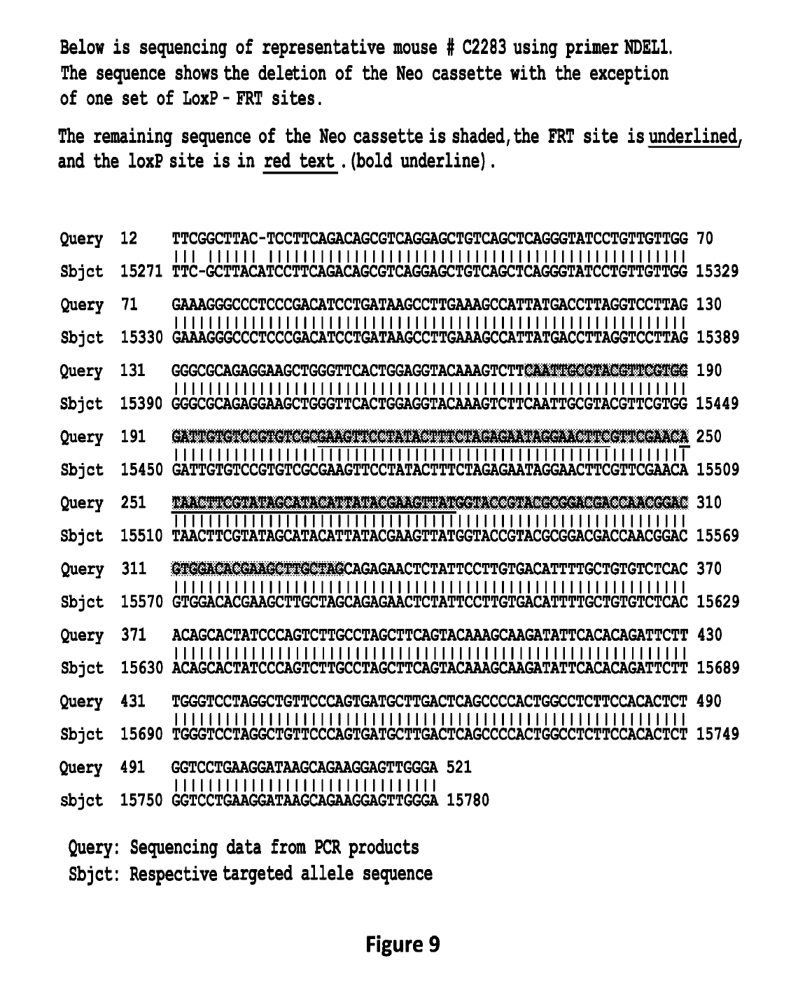

Primers NDEL1 and NDEL2 are used to screen mice for the Neo cassette deletion. (FIGS. 8A, 8B and 8C) These mice are identified in Tables 1, 2 and 3 (heterozygeous mice), and Table 4 confirms that homozygous mice screened and selected for furtherance of producing transgenic mice of the invention.

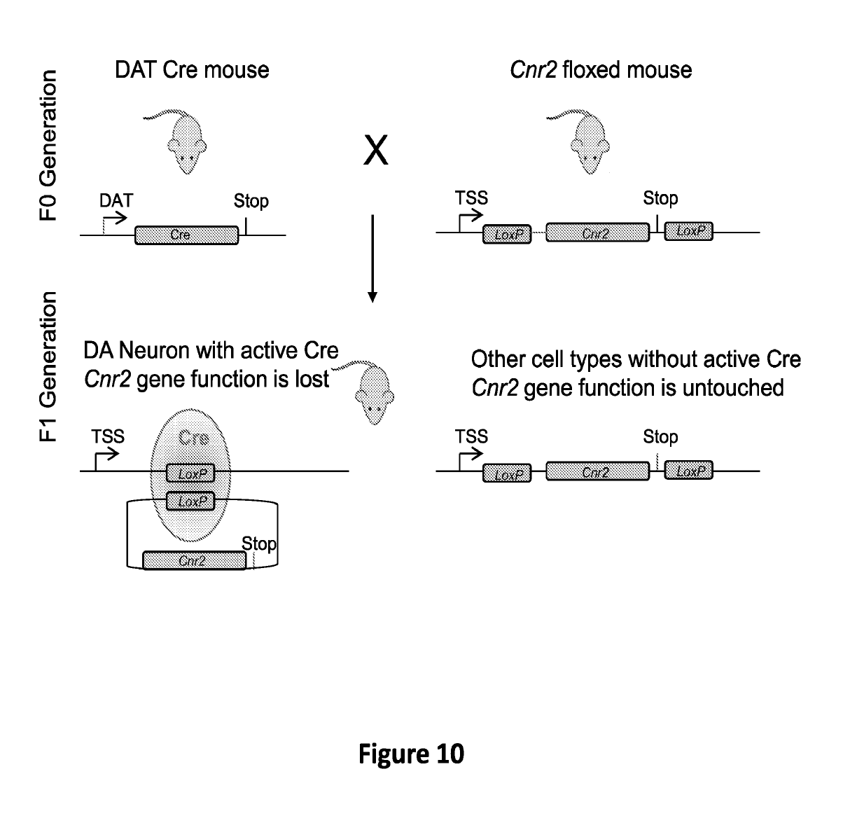

Once the homozygous CB₂ flox mice are produced, breeding of the Cnr2-floxed mice with various Cre recombinant mice takes place. FIG. 10 provides a schematic of the production of the Cre mice. Basically, a Cre mouse is bred with the loxP (floxed) mouse. The resulting CreLoxP mouse is the F₁ generation in FIG. 10. Then, these mice are screened, and F₂ generation mice are produced from the various Cre mouse models used for breeding.

The Cre mouse is an example of a mouse system that consists of a single enzyme, Cre recombinase, that recombines that sequence without having to insert any extra supporting sequences. Another system that is useful for such creations is the FLP-FRT recombination system. Those of ordinary skill in the art are well aware of other such systems.

Mice generated by this procedure and that have the Cnr2 gene floxed are provided and tested to ensure the requested DNA is present. As such, genotyping of these mice is conducted. Tail samples of DNA tissue are ways in which to obtain tissue for such sampling. Other mechanisms to obtain DNA samples also are useful. Biopsies of ears are also useful for genotyping.

A typical master mixture for preparing a DNA sample for PCR amplification is provided in the following examples. Those of ordinary skill in the art are familiar with the mixes useful to prepare DNA samples for PCR.

For example, Southern blots, restriction fragment length polymorphism or RFLP analysis, and/or Hederoduplex Analysis (HA) and/or Conformation Sensitive Gel electrophoresis (CSGE) are other genotyping methods.

Further, gel electrophoretic studies are conducted on the PCR resultant DNA to determine what genotypes of the various transgenic mice produced.

The resultant transgenic mice of the invention are then evaluated. For instance, CB₂R is tested for the behavior effects of dopamine, DAT-Cnr2. Anti-inflammation and neurodegeneration are studied when known agonists of synthetic cannabinoids are tested in Dat-Cnr2 and Cx3crl-Cnr2 mice of the present invention. Examples of tested compounds include JWH13 obtained from Tocris Bioscience. An animal mouse model useful in identifying reduced hyperalgesia in multiple sclerosis is another animal model produced by using the transgenic mice of the present invention.

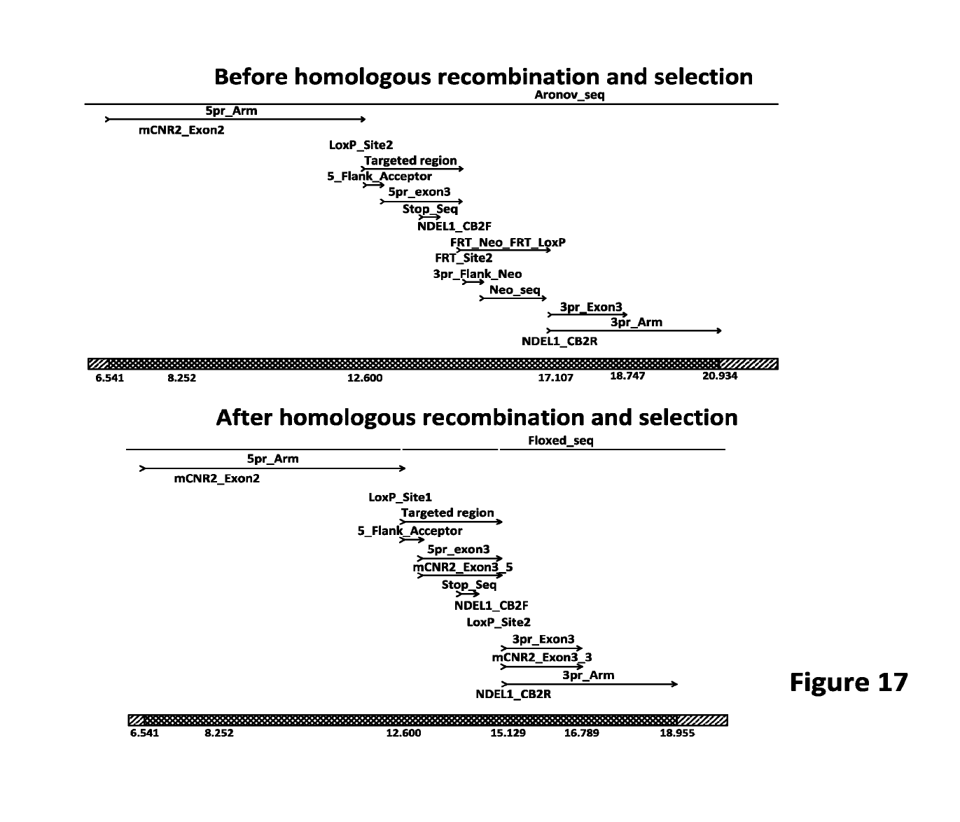

The transgenic mice of the present invention have the DNA sequence provided in SEQ ID No: 1, provided herewith below. Additionally, FIG. 17 provides the clone constructed with the replaced Cnr2 gene having the LoxP sequences flanking the Cnr2 coding region. This construct is useful in any embryonic stem cell delivery for the production of transgenic mice.

Final Cnr2-floxed mouse sequence (entire CB2R protein coding sequence and splicing acceptor site (AG) are sandwiched by loxP sequence for homologous recombination).

### Sequence Listing

SEQ ID NO: 1. Key: Shade: exons; Underline: loxP sequence; Bold: FRT sequence; Italics: restriction enzyme site engineered; Double Underline: splicing acceptor site sequence; Broken Underline: residue Neo cassette sequence.

AAACAGTGTATCCAGGCCACCACCGATTGATCAGGGCCAGAGAAAC
AGACCCAGCAGCTGACCTGCCACCCCGAGCCAGAATACTACAGAGT
TTTTAAGCCCAAAATCCACAATCATCTGTGCCAAGTTACCCCACCAG
TCAGGATTTAGGGATAGGGGACGTCCTTAGGAACATGTCTTTGTGGT
ACACCTATCCTGCTCCCATTGGTTGGGGTATTCAGCAGTGGCAGGGG
ACTTGCCTAGCATTCATGTCTCAACTTGACAGCTAGGATGTCCGTTA
CCAAGGGAGCTGCTGGGACTTATACTTTATTTGACCCCTATACAAAG
TGGAATGGCTTTTATTTGGTTCCTTCAATAATAATAATAGGAGGAGG
ATGAGAAGTTCGAGGATGGCTTTGAACATATTTTGAGTTCTAAAATA
GCCTGGACTAGATGTAGCCCTGACTCCGAAAGGACATGGCTCCGTGG
GCGAAAGGGCTTGGCACACAAGCCTACTGACCTGATTTCAGTCCCCC
AGATGTAGTCATAATAAGTGATACATGAGTAAAATTTAAAGCGAAA
AGTACTAGCAATGATAATAATAAATAAAACAAAATAAAAAATAAAA
CATGATTTTTTTTTCTCCTGAAAAGATGTATAGGCATTAGGTTTATA
TAGTTAGTTAGTTAGTTAGTTAGTTAGTTAGTTAGTTAAGACAGGTG
TGGTTGTGTTTAGTTAAGGCAGGTGTGGTTGTGTATGCCTTTAATCT
CGGCACTAAAAGAGGATTAAAGGAGACAAGGCAGGTAGATCTCTGA
GTTCAAGCCTAGCCAGGTTTGCAGAGTGAGTTCCAGGACAGCCAGG
GCTACACAGAGAAACCCTGTCTCAGAAAACCAAATAGATGGATAGA
TAGATAGATAGATAGATAGATAGATAGATAGATAGATAGATAGATA
GATAGATAGATAAAATAGTTTTAAAACATTTATTTACATTTATTTTT
ATGTGTATAACTGTTTTGCTTTCATGTATGTCTGTGTACCGTGTGTG
TGCCTGGTGTTAGCAGACAGCAGAACTGGAATTAAAGACAGCTATG
AGCTGCTATTTGAGTTCTGGGAACCAAACCCCAGTTCCCTGCAAGA
ACGGCCAGTGTTGTTAATCTCTAAACCATCTCTTCCAGCCCCATGC
ATTTGGTGTGTGTGTGTGTGTGTGTGTGTGTGTGTACTATTAATTGC
TTTTGAGACAGGGTCTCACTATGTAGCTGGCCAGGAACTTGCCACA
TAGAACAGGCTGTCCTCAGACTCATAGAGATCTAGCTGCCTCTGTT
TCCCATGTGCTAAGATTAAAGCTGTGTGCCACCATAGCAAGCTGGA
AAGGTCTTATATACACTTTGAAAGGATCAAAAAGAACTTGCCAGTT
CCCAGTTTCAAAAACTAGAACGAATGTCCTCGGTGCGCTTGGCCTC
CTTAAGAATGGGGGGGGGGTATTGTTATTGTCTCTTCACAAGTGAG
AAGAGGGACTTGCCCAAAGTCACATGATGAGAGTGACAGCATTGG
ACCCAGAGCAGCTACTTATACATCAAACACATCCTTGCCCTAGAAA
TAGGTCTTCTAGAAGGCACCCATGTGACTTGCAGAGGGTATCTCTA
TCTTCGTGGAGACAGGGAGCCGGGCTTCCTGTTGCTGTGTGCATC
CTGTTGTTCTCTTGTTAGGATGTCCATCAAATGCATGCATTT

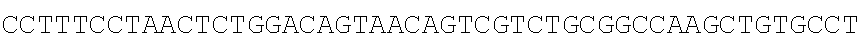

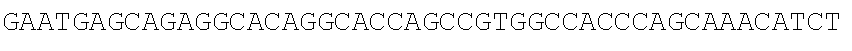

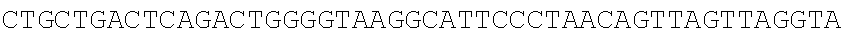

GGTAGGTAGGTAGGTAGTTTGAGACAGAGTTTTGCTATGTTGTCT
AAGCTGGCCTTGAACTCATTATGTTACCCAACATTAAACTTACAG
CAGCCCCAGCCTCAGAATTTACTAGATTCTGACACAGAGCAGTCT
GGCCTCAGCCTCCTGAGTGCTGGGATTACAGGTATATGCCACCAT
GCCTGGCAATCCCTACCAGAACTTTTATTTATTTATTTTACTTAAA
AAAAAAACTTTTATTTATTTTTTAAATTTTTAAAAATTTATTTTAT
TTTTATTTACTTACTATTAGTGTGTGTGTGGGGGGGGAAGATGTA
TATGTGGGTGTGGGTGCATGTATCAGATATCGTGTGTCCTGCTCT
GCTGATGTCTAACGTACTCCTTTGTAACAAGGTCTCTGGCTGAGC
CCAGAGCTAGGCTGGCAGCCAGCAAACTCCAGTGAGCCTCTGTC
TGCTCCAGACAGTGCTGAAGTTATGGGTGTGTGTGTGGCCAGGCC
CAACGTTTTATGTGAGTGCTGGGAACTAACTCAGGTCCTTAAGGAT
GTCTTATCCACTGAGCCATCTCTCCAGCCAACACCCTTGCAACTTG
ATTTCTTATATTATAGTGTCATGTGGATGAAATAAAATTATGACT
GGGAATAATATTAATTGTATCAGAGTTTTATTTACGTATTTTGTGT
GCATGTGTGTATGTATGCATGTGTGTGTGCATGTCACAGCTGGTTT
CTGGGAGCCAGAGGACAACTTGCTTTGTTGGCTCTGTCCCTCCATC
TTTATTTGGGTCCAGGGGTTCCAATTCAGGTTGTCTGGCAGGCACC
TTTACCAACTAATCTGTCTCTCCAGCCCCAGGAGCAGCAGTCTGCA
GAAATTAAATTCCATACTCTCAGTTATTGATGTAGTTGAGGGGCAG
GCAGCATGTAAAACTGCTGGGGAGCTGCTGCATGGGGGAGGGGGG
GGCTTTGGAAGGCTCCACTGGGCAGAGAAGCAAAGGTAAGTCCCG
AAGGGCTATGAAGCCAGAGGCCAGATGGCATGTTTTTCCAGAAGC
AGCAGATGGCCAGCTGGGTGGGGCTTGGGGGCTAGAGCGGCCCTG
GGTACACGCCTTTGTAGCACAGTGCACTGCTTTGAACCCTTTATGT
TTAGGCAGCCCAGTGCACTGCAGTGGGCAGTGTACTGAGCTGCCC
CAAAAACACACAACAGAAGTCCTACATGTCACTGAAATTTTCCCT
GTCTACATAGGGATGTGGAAATACACACACACACACACACACACA
CACACACACACACACTCACACACATACACACACATGCATTCACAC
ACACACAGAGTCTATTCTGAGTTAAAAAATAAGGAAAAATATGTC
AGTGGTAAGGCTTTTGTGAATGTGCAAGGCCTTAGATTTGATTCCC
CAGCACCACAAAAATAAATAAATACAAAACAGAGCTGGGCAGTA
GTGGTGCACACTTGAGAGGCAGAGGCAGGTGGATGCCTATAGTTT
AAAGCCAGTCTGATCTACATAGAGAGGTCCTGTCTCAAATAAATA
AAAAACGAACAAATAAGAACCAAGAACAAAATAAAATACATTAA
ATGTGTATATCGGTTTATCAGCTCACAGTGTCTGACTTCCTCTCTT
CCTTCCTATGCTGAATTTCCTCCTGCTAAAAGATAAGTGAAAATT
TTTTAAAAGGTCTCTTAGAGGACAGTTTTATTTGGGGGGAGTTGA
TTGGTTTGTTGGGTACAGATAAAACTAGCCCTTGCTGGCTTTGAA
CTCCATATAGACTGCAAGTTATTTATTTATTCATTGATTTTTGAGA
CGAGGTCTCCCTAGGCCATCAGGACTGGGCCTGAACTCAGTCAG
TCTGTAATCCGGACAGGGCTCCTGAGTCGCTGAGACACAGACCT
AGTCACCAGGTGTCCTCCTCTTCCTTCTATGTGGGTTAATTTTGA
AATAATACTTATTGCCTTTATTAATCCCAGCCGTGGGAGGCAGAA
GCCGGTGGATCTCTGAGTTCGAGGCCAGCCTGGTTTACAAAGCAG
GCTCCAATACAGCCAGGACTACATAGAGAAATTCTGTCTTGGAGG
AAAATAAAAAAAAAAGCTGGGCAGTCTTGGCTCGTGCCCTTAATC
CCAGCACTAGGGAGTTTAGGGCCAGCCTGGTCTACAGAATGAGTT
TCAGGATAGCCAGAGATGCATGGGAAAACCCTGTCTCAGTAAAA
CAAAATTATATTTGTTTATTTATCCATTCATTTATTCTCGTGTGTG
TGTGTGTGTGTGTGTGTGTGTGTGTGTGTGTGTGTGGTGGTGTGT
TGGGGAAGATGCCATGGTACGCGTGTGGAAGTAAGAGGACAATTT
GCATTCTACCACATGAAGCCCAGGGATTAAACTCAGTACATCAAG
AGTTTTTCTCAAGCGGGGGGTGGTGGCGCACGCCTTTAATCCCAG
CACTGGGGAGGCAGAGGCAGGCAGATTTCTGAGTTCAAGGCCAG
CCTGGTCTACAAAGTGAGTTCCAGGACAGCCAGGGCTATACAGAG
AAACCCTGTCTCGAAAACCAAAAAAAAAAAAAAAAAAAAAA
AAGTCTTTCTCTACCTGTGGAGCTATCTCAATCACCTCTGGCCTTT
AGTTAGTTAGTTAGTTAGTCAATTAGTTAATTTTAGAGTTTTGGGG
TAGCATTGGCTGTCCTGGAACTAGCTCTGTAGACCAGGCTGGCCTT
GAACTCACAGAGATCCACCTGCCTCTGCCTCCAGAGTGCTGGGAT
GAGAGGCCATGTGCCACCCCTCTCGGCCTTTCTTTTAATTCTTTAT
AAAGTATGCCCAAGAGTTGCATAAGTCTCTTGCAAATGGGAGCTA
TAACCCCTCCAGCAGACATCTGAGAACTCTTGCTTGTTTGTTGATC
ACTCCCGGGCTTATAAGGTCAAATTTCATGCCCCCTAGATAGAAT
ATTTCCACATAACCTGTTTAGAAAATTTGTCTTTTTAGAAAGATGT
TTAATTTGCTATCTTGTGAAGTTTACTAATGCTTCATACAATATTC
ACTTGTTCTAAGTTTTTTCTTCCAAATCCAGGACATGCTTATATTA
AAAAATGATTCTTTATGTGATGTTTAAATAAATGGGACTTCTATAT
CTTATCTAGTACAGTTACTTTTTAAGACAGAGTTTCACAGAACCAA
AGATGACTTTGACTCGACCCCCCTGCCTTCACCTTCCAGGTGCTGT
GCTTGAGCTACGTTCCCTACAGGAGTATAAAAAAAGGTTGTGTTT
GAGGCAGGGATTTCCTATGTAGCCCAGGCTGGCCTACAAACTCAC
AGAGATCTGTGTATGTCAGCCTGCCGAGTGCTGGGATTAAAAGCA
TGCACCACCACTCCATAAAATATTTATCTTTTGAGTTTATCTTCCC
AACACTTGAGGTGAAACCAAAGTATTGGCTCCCATCCCCAAACAG
TTACTAAGCCTTTAGTATCTTACCAAAGCTGGAAGAACACAGAAAT
CAAAATGAGATATAAGCCAGGTTTGGTGGCGCATACCTGTCTGGAA
TCCCAGCTGCTTTGAAACCTGAAGTAAGAGGGTCTACCTGTGAGAC
CATGTTTCAAAAAGCAAAAGGGAATCTGGCCAGCAAGATGGCTT
TGAGAGTAGAAGTGAGCAGTAGGAGGCCTGAAGGCCCGAGTCC
AAATCCCAGAACCCCTGGTAGACCTCCACACACACTCTGTGGCA
CACTCATGCCTGCAATACGCATCACATGTGAGCACATATGTGT
ACAGACACACACTAACAACAACAACAACAACAATAACAATAAT
AGAAAAACAATATCCCTACATCTTCAGTTCCACTTCTAGGTT
TTGATCTCTTGGCTGGTGGCCCCTAGGAATTTGCATGGC

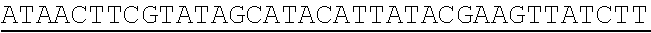

TCCCCTGGGTTTCAAAGGGAAGTTTCAGGATTTATCTT
TTTAGAAAGCAAAAGACCAAAACATCTTGCAAGTTACTTC
TGCTTGTTTGGAAACAAAAAGACTTAGCTTGAGGAAAAAAGG
TAAATGCTTGGCAGCAAACTATAAATGGATATTTTTGGGGTTGGTG
AGATGGCTCAGCAGTCAAGAGCTCTTCCGAAGGTCCTGAGTTCAAA
TCCCAGCAACTACATGATGGCTCACAAACCATCCATAATGAGATCT
GACGCCCTCTTCTAGTGTGTCTAAAGACAGCTACAATGTACTTACA
TATATAATTATAAATAAATCTTTGGGCTGGAGCAAGTTTGCATGGC
ATAACTTCGTATAGCATACATTATACGAAGTTATCTTTCCCCTGGG
TTCAAAGGGAAGTTTCAGGATTTATCTTTTTAGAAAGCAAAAGACC
AAAACATCTTGCAAGTTACTTCTGCTTGTTTGGAAACAAAAAGACT
TAGCTTGAGGAAAAAAGGTAAATGCTTGGCAGCAAACTATAAATG
GATATTTTTGGGGTAGAGTGAGCAGAGGCCCTGAGTTCAATTCCCA
GCAACCACATAATGGCTTACAACCATCTGTACAGTTACAGTGTAAT
CATATAAATAAAATAAATAAGTCTTTAAAATTTTTTTTGTGTGTGC
TGGTGTAGTGTTGGCACATGCCTTTAATCCCAGCACGTGGGAAGC
AGAGGCAGGCGGATTTCTGAGTTTGAGGCCAGCCTGGTCTACAGA
GTGAATTCCAGGACAGCCAGGGCTACAGAGAGAAACCTTGTCTTA
AAAAACCAAAAAAAATTTTAAAAATTAAAAAATAAATGGATATTT
TCGGCACCTGGGTAAATGCTTGATTGGTGTCAGCTCTCAGTTGAC
GTCATCACCTGTTAACATTCAAGGATTCTTTTCTCCTTGCCCA

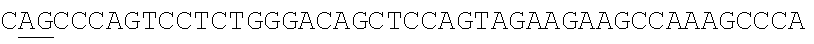

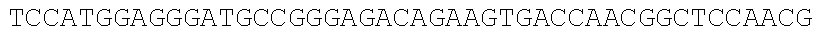

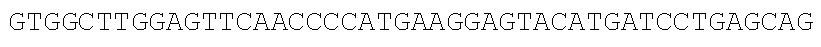

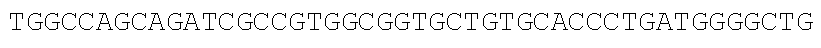

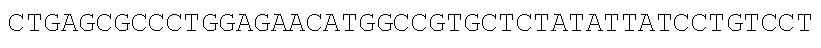

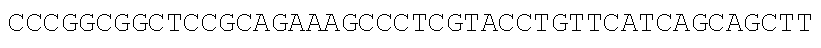

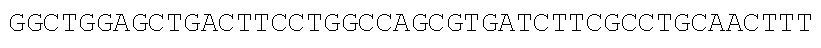

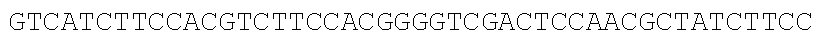

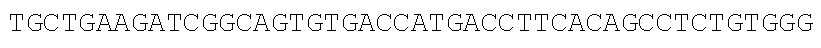

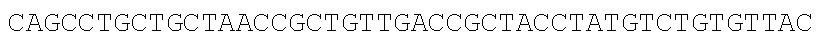

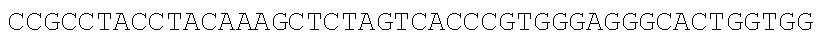

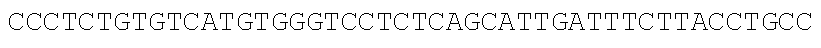

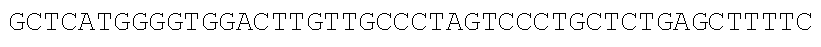

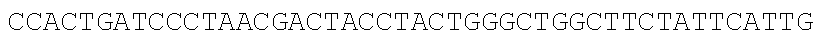

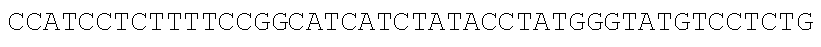

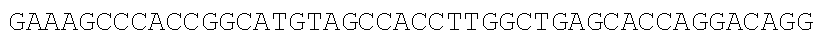

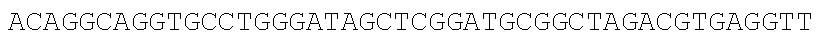

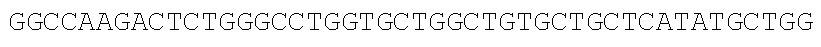

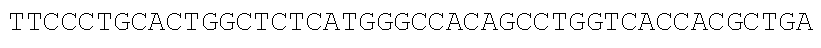

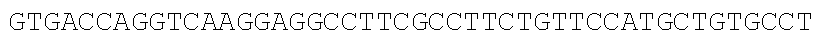

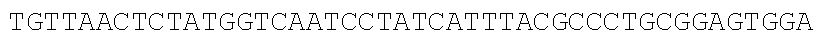

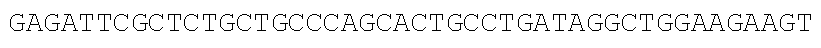

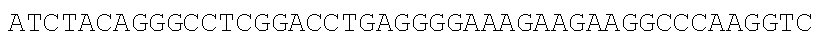

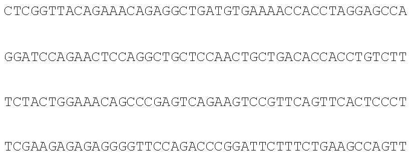

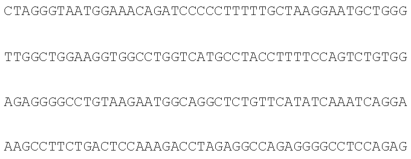

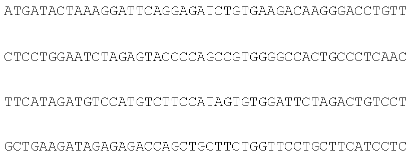

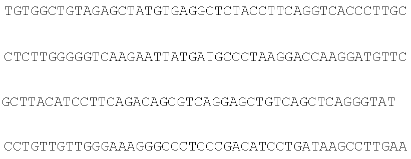

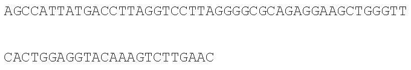

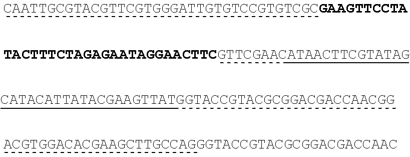

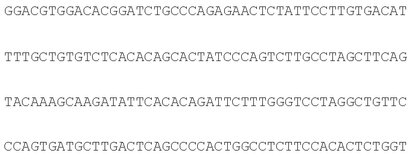

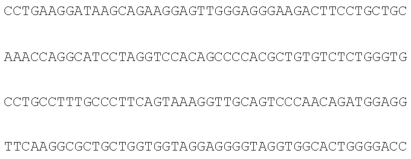

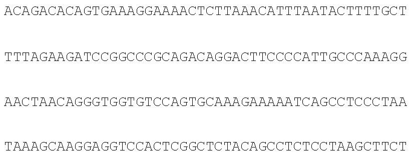

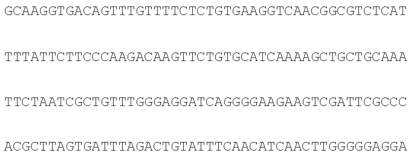

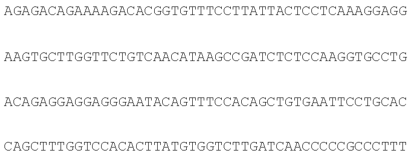

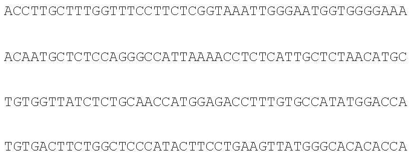

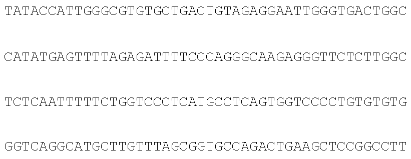

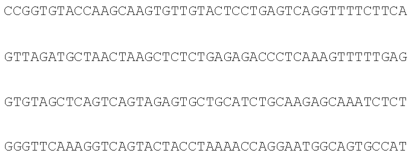

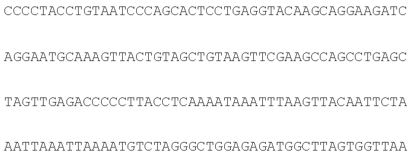

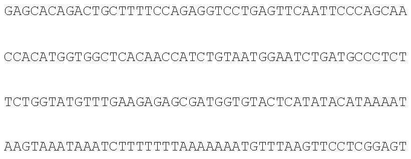

TCTTGTTAAGTTAAGTTCAACGGACAAAAGATAGACTAAAA
TAACCACGTCTGCCCTTAATCTTTCAGCAGCCATTTCTCTC
CCTCCGCATAAAGCTCCTTCTAACCTTGTTATTTCTCAATCTCT
TCGGCAACTCTCCAATCTGCTAAACTCCACCTGCTGGCTGGAG
GTATAGCACAGAGAACCCGCGGCTCCTAAATAGTCACTAAATAAA
ACGACCTGGCTTCAGCCTTCCCCTGAAGATTGGTGCAACAACGTGG
ACTTCTCCCAGTTTTCCCCACACCTTGGGTCTTTCTCACTTGCGGG
GAATCCGGCAATTAAATCCTTAGGCGAGTCTACGGAATTTAAATTC
AATAGAGCAGAACCAGCAACGCCGCCTTAAGTCTTTCTCCCTTCCA
ATGCCAGAGGGCGCTCCAGAGCCGCAAGCCTGACCCCACCCCCTCT
CCTTCCAAGCCAATCGTTACTCTGAGAGGCGGGGCTACTCGCCGGC
GCTTGCGTGTTAAGAGGGGGGCGGTGCGGGCCCGGAAGCAAGGATG
TAGGAGCTGAACTTGAGTGGTGGGCTGTGGACTCTGGGATGCTGCT
GCTGCTGCTGCTGCTGCTAGTGGCTGCGGCCCAGGCAGTGGCCCTG
GCTCCGCGCCGCTTCACTCCGGACTGGCAGAGCTTGGACTCGCGGC
CACTGCCGAGCTGGTTCGATGAGGCCAAGTTCGGGGTGTTCGTGCA
CTGGGGGGTGTTCTCGGTGCCCGCCTGGGGCAGTGAATGGTTCTGG
TGGCACTGGCAGGGCGATCGGATGCCGGCCTACCAGCGCTTCATGA
CAGAAAACTACCCGCCCGGCTTCAGCTACGCCGACTTCGCACCGCA
GTTCACAGCGCGCTTCTTCCACCCGGATCAGTGGGCCGAACTCTTT
CAGGCTGCCGGGGCCAAGTGAGTGCGGGACGGGCGCGAGGCGGGA
GCAGGCAGGCCATAGCCGGCGCTGCCTGACCTCGGAAGGTCCAGGA
CCCTACGTTCTGCATCTCGGAGGAAGGACGCTAAAGACGAGAATCC
AGCTGAGGATCAGTTAAAAAAATAAATAAATAAAATAAAATAAAAT
AAAATTAAGGCGGGAAAGCACTGGGTGGTACACCTGTGCGGAGAGA
GACTAGAATCAGGCTACATCGGTGTCTCGTTTGAAAGGTCCAGAAC
ACAGCCCAGATTCCCGTCGGTGGCCCTGAGTTGTAGATCTGACCAG
TCCCCTTAGGCTACGGGTAGCGTCCGTCCGTGGAAGCTTATAAAAA
AGCGCCGCCTGACTTCCTTGTAGCCCTGTGCCTCTAAAGGAAGAAG
GAAGGATGGAAGTTTGGGGTTGCTCTTTGTTGGGGTCTCCCTCACT
TCTCACTTGCTTATAGTAGAACTCAGAGGAGGAAGGGGGTTGTTT
GGGCAAAGCCCTGCAGAGGAAGTGCGTTTTGAGAGCCCTCTAAGC
CAGAGAAAATATTGGCTGACTTTGGACGCGGGATTTGGCCACTGTG
AGGGCAGCCTCCCGCTCCTGGCTTAGTGTAGTCCTCCGCTCTCTTC
ATTGATTTTCTGCCAAACTCCGACTTTTTATTCCCTCGGGACGTTA
GGTTTTCTCCCCCGAAGGGAAAGGCTGTGTATAGCTGGCACTTAGT
ATTTTCGCCAAGTAAAAGGCCTGAGGGAACAGAAATCTGTCATCTT
GCCATCTTGAGAGCCGTAATTTACAAAGTTGAACTCCAGCCTTTTT
CCAGACTTCCTGTTTAATGGACGAGGTAACTAGCCCAGAGAGAGG
AAGGAGAGGAAGGAGCCTGGTGTCCTAGACTGCAGGTCTATGATC
TGGAAGTGAAAGCTCCAGAAAGCTCATGGGACAGGTGTGCCCAGG
CTCTGCAAGCCATACCCAGAGGGCGTGGTTATTTGCTTGTTGAGTG
GGAAGGGATTTTATATTTCAGAAGGACTTTTTGAAAGGGGAAAGA
GATGTATTGCAGGAAGGACTGATGAAGTTCAGGGGCCCCTACACC
AGCTAGTGGTAAAATCTCATGTAACATGCTATCTTTTCCTATAAAA
GTAAGTGATATATTGAATGGACTGATTATCTCAACACATTTTTTTT
TTCAGTCCTAGATATTTGAGCCTTTGTCACCTTAAATGACAGGCA
GTTTCACAATCTTGGTTAAGAAAACAAGCA

The construct (FIG. 17) of 5′ and 3′ arms are for recombination to delete the targeted sequence including open reading frame of exon 3 and its splicing site. Neo cassette which is an antibiotic gene for the drug selection of positive embryonic stem cells successfully transfected with the construct. FRT flanking Neo cassette enables deletion of the Neo gene by mating the version 1 of Cnr2-floxed mice with recombinant flipase expressed transgenic mice. The resulting version the 2 Cnr2-floxed mouse contains loxP sequence flanking the CB2R entire coding sequence and 5′- acceptor splicing site without Neo cassette.

The procedures for the creation and generation of Cnr2 cannabinoid receptor transgenic gene floxed conditional knockout mice useful in an animal model for basic and drug developmental research is provided in the examples, but is illustrative thereof and not limitative of the invention.

### EXAMPLE 1

The construct (new FIG. 17) of 5′ and 3′ arms is for recombination to delete the targeted sequence including the open reading frame of exon 3 and its splicing site. The Neo cassette as antibiotic gene for the drug selection of positive embryonic stem cells successfully transfected with the construct. FRT flanking Neo cassette enable deletion of Neo gene by mating the version 1 of Cnr2-floxed mice with recombinant flipase expressed transgenic mice. The resulting version 2 Cnr2-floxed mouse contains loxP sequence flanking the CB2R entire coding sequence and 5′- acceptor splicing site without Neo cassette.

Targeted iTLBA129/SvEv×CSBL/6 hybrid embryonic stem cells are microinjected into C57BL/6 blastocysts. Resulting chimeras with a high percentage agouti coat color are mated to C57BL/6 FLP mice to remove the Neo cassette (for antibiotic selection of recombinant clone) resulted. The coding exon of Cnr2 are flanked by left LoxP at 5′-splicing site and right LoxP downstream of the stop codon so the Cre recombination produces cell-type specific deletion of the entire Cnr2 coding region and splicing site result. This is the first conditional Cnr2 full knockout mouse. See FIG. 2 for schematics of mouse development of the invention.

LoxP sites; Rectangular: for recombinase Cre to delete the target Cnr2 protein coding and splicing sequences. FRT sites for recombinase flipase deletion of drug selection marker Neo; LA (long arm), MA (middle arm), and SA (short arm): genomic regions for homologous recombination. (See FIG. 2)

### EXAMPLE 2

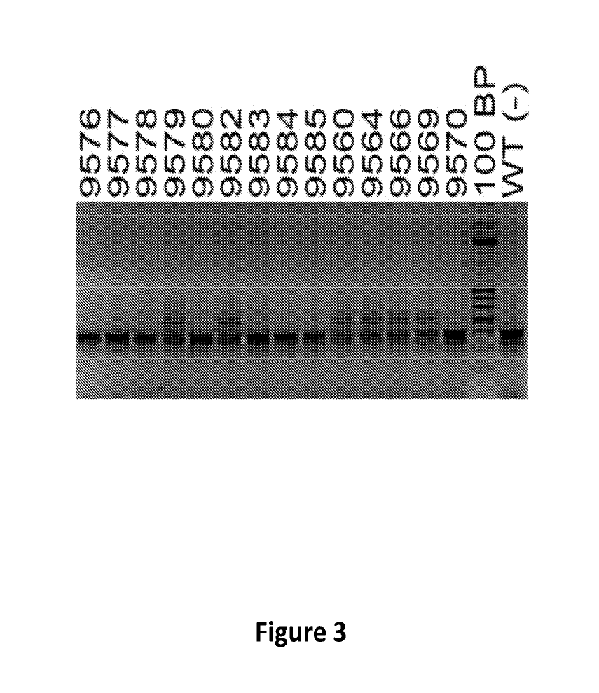

Primer set NDEL1 and NDEL2 is used to screen mice for the deletion of the Neo cassette. The PCR product for the wild-type is 386 bp (lower band). After Neo deletion, one set of LoxP-FRT sites remain (˜159 bp). A second band with a size of 545 bp (upper band) indicates Neo deletion (9570, 9582, 9560, 9564, 9566, and 9569). (See FIG. 3)

After a 2 minute hot start at 94° C., the samples are run. The PCR product is run on a 2% gel with a 100 bp ladder as reference. Tail DNA sample from a FLP mouse is used as a positive control and is denoted by a (+) in the gel photograph.

### EXAMPLE 3

EconoTaq Plus Green 2× Master Mix (Lucigen catalog #30033-1)
11.00 μL ddH₂0
12.50 μL EconoTaq Plus Green 2× Master Mix
0.25 μL 100 M Primer
1.00 μL DNA

After a 2 minute hot start at 94° C. the samples were run using the above conditions. The PCR product was run on a 2% gel with a 100 bp ladder as reference.

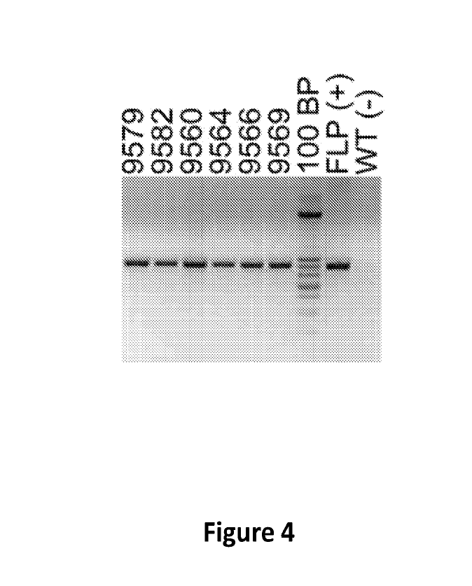

The presence of FLP (flipose) in Flp mice: Primer set FLP1 and FLP2 (obtained from Ingenious Targeting Laboratory, hereinafter “iTL”) is used to screne mice for the presence of the FLP transgene in Neo-deleted mice. The amplified product for primer set FLP1 and FLP2 is 725 bp (See FIG. 4)

### EXAMPLE 4

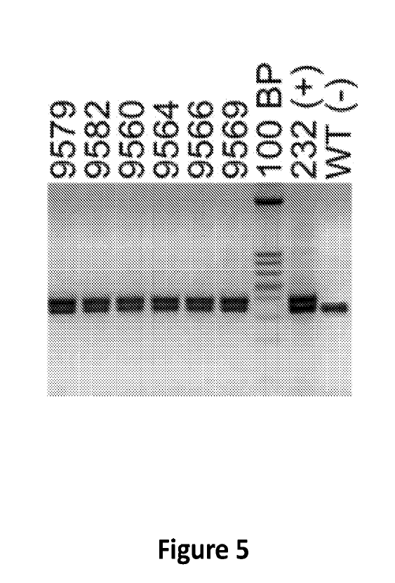

A PCR is performed as in Example 3 to detect the presence of the distal LoxP site flaking coding exon using the SC and SDL2 primers (from iTL). This reaction amplifies a wild type product 350 bp in size. The presence of a second PCR product 44 bp greater than the wild type product indicates a positive LoxP PCR in Neo-deleted mice. (See FIG. 5)

### EXAMPLE 5

Confirmation of Short Homology Arm Integration

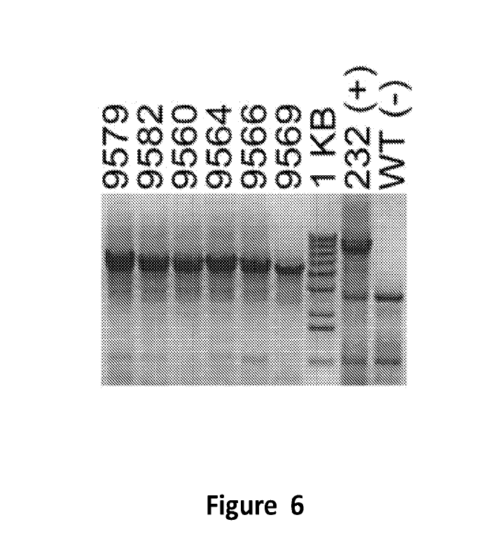

Tail DNA samples from positive mice are amplified with primers NEO-GT and A1. NEOGT is located inside the Neo cassette, and A1 is located downstream of the short homology arm, outside the region used to create the targeting construct. NEO-GT/A1 amplifies a fragment of 4.34 kb in length. Due to the presence of the Neo cassette in the expanded ES cell, the amplified size is 6.3 kb. (See FIG. 6)

### EXAMPLE 6

Somatic Neo Deleted Mouse Information

The following heterozygous mice are confirmed for Somatic Neo Deletion. (See Table 1)

TABLE 1

| Mouse # | Sex | DOB | Clone # | Parent Info |
|---|---|---|---|---|
| 9579 | F | May 21, 2014 | 232 | CH × C57BL/6 FLP |
| 9582 | F | May 21, 2014 | 232 | CH × C57BL/6 FLP |
| 9560 | M | May 21, 2014 | 232 | CH × C57BL/6 FLP |
| 9564 | M | May 21, 2014 | 232 | CH × C57BL/6 FLP |
| 9566 | M | May 21, 2014 | 232 | CH × C57BL/6 FLP |
| 9569 | M | May 21, 2014 | 232 | CH × C57BL/6 FLP |

CH is chimera mice and CS7BL/6 is the mouse strain that expresses flipase (FLP). The CH chimera mice are used for the fur color selection of Neo, Somatic and germ line deletions.

### EXAMPLE 7

Absence of FLP Transgene

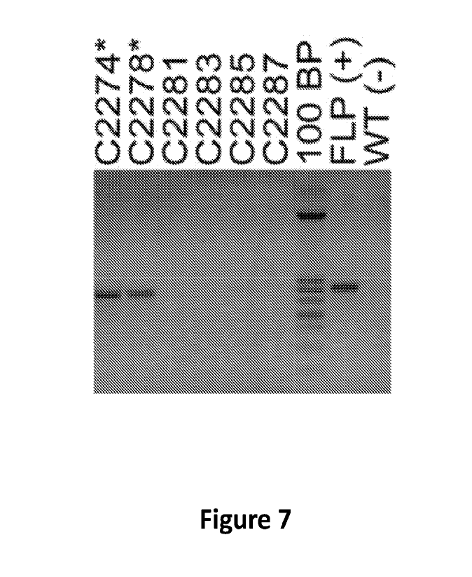

Primer set FLP1 and FLP2 (obtained from iTL) is used to screen mice for absence of the FLP transgene. The amplified product for primer set FLP1 and FLP2 is 725 bp. (*Mice C2274 and C2278 are FLP present and are sacrificed.) (See FIG. 7)

### EXAMPLE 8

Screening for Homozygous Neo Deletion with LoxP Flanking Entire Cnr2 Coding Region

Primer set NDEL1 and NDEL2 (obtained from iTL) is used to screen mice for the deletion of the Neo cassette. The PCR product for the wild-type is 386 bp. After Neo deletion, one set of LoxP-FRT sites remain (159 bp). A second band with a size of 545 bp indicates Neo deletion. A single band of 386 bp indicates a wild type mouse; two bands 386 and 545 bp in size indicate a heterozygous mouse; and a single band 545 bp in length indicates a homozygous mutant mouse. (See FIGS. 8A, 8B and 8C)

### EXAMPLE 9

Germline Neo Deleted Mouse Information

The following heterozygous mice are confirmed for Germline Neo Deletion and FLP absence. (See Table 2)

TABLE 2

| Mouse # | Sex | DOB | Clone # | Parent Info |
|---|---|---|---|---|
| C2281 | M | Jul. 23, 2014 | 232 | SND # 9566 × C57BL/6 WT |
| C2283 | M | Jul. 23, 2014 | 232 | SND # 9566 × C57BL/6 WT |
| C2285 | M | Jul. 23, 2014 | 232 | SND # 9566 × C57BL/6 WT |
| C2287 | F | Jul. 23, 2014 | 232 | SND # 9566 × C57BL/6 WT |
| C2302 | F | Jul. 23, 2014 | 232 | SND # 9560 × C57BL/6 WT |
| C2303 | F | Jul. 23, 2014 | 232 | SND # 9560 × C57BL/6 WT |
| C2304 | F | Jul. 23, 2014 | 232 | SND # 9560 × C57BL/6 WT |
| C2377 | F | Aug. 4, 2014 | 232 | SND # 9579 × C57BL/6 WT |

The following heterozygous mice are confirmed for Somatic Neo Deletion. (See Table 3)

TABLE 3

| Mouse # | Sex | DOB | Clone # | Parent Info |
|---|---|---|---|---|
| 9579 | F | May 21, 2014 | 232 | CH × C57BL/6 FLP |
| 9582 | F | May 21, 2014 | 232 | CH × C57BL/6 FLP |
| 9560 | M | May 21, 2014 | 232 | CH × C57BL/6 FLP |
| 9564 | M | May 21, 2014 | 232 | CH × C57BL/6 FLP |
| 9566 | M | May 21, 2014 | 232 | CH × C57BL/6 FLP |
| 9569 | M | May 21, 2014 | 232 | CH × C57BL/6 FLP |

### EXAMPLE 10

Germline Homozygote Neo Deleted Mouse Information

The following homozygous mice are identified. (See Table 4)

TABLE 4

| Mouse # | Sex | DOB | Clone # | Parent Info |
|---|---|---|---|---|
| C2626 | M* | Sep. 26, 2014 | 232 | ♂GND #C2285 × |
| | | | | ♀GND #C2303 |
| C2627 | M* | Sep. 26, 2014 | 232 | ♂GND #C2285 × |
| | | | | ♀GND #C2303 |
| C2632 | F | Sep. 26, 2014 | 232 | ♂GND #C2285 × |
| | | | | ♀GND #C2303 |
| C2643 | M | Sep. 29, 2014 | 232 | ♂GND #C2285 × |
| | | | | ♀GND #C2304 |
| C2645 | M | Sep. 29, 2014 | 232 | ♂GND #C2285 × |
| | | | | ♀GND #C2304 |
| C2648 | F | Sep. 29, 2014 | 232 | ♂GND #C2285 × |
| | | | | ♀GND #C2304 |
| C2671 | M | Sep. 28, 2014 | 232 | ♂GND #C2283 × |
| | | | | ♀GND #C2302 |
| C2672 | F | Sep. 28, 2014 | 232 | ♂GND #C2283 × |
| | | | | ♀GND #C2302 |

Primer set NDEL1 and NDEL2 (FIG. 2) is used to screen mice for the deletion of the Neo cassette. The PCR product for the wild-type is 386 bp. After Neo deletion, one set of LoxP-FRT site remains (˜159 bp). A second band with a size of 545 bp indicates Neo deletion. The presence of the Neo cassette is not amplified by this PCR screening because the size is too great.

A PCR (as in Example 8) is performed to detect presence of the distal LoxP site using the SC1 and SDL2 primers (iTL). This reaction amplifies a wild type product 350 bp in size. The presence of a second PCR product 44 bp greater than the wild type product indicates a positive LoxP PCR.

EconoTaq Plus Green 2× Master Mix (Lucigen Catalog #30033-1)
11.00 μL ddH₂0
12.50 μL EconoTaq Plus Green 2× Master Mix
0.25 μL 100 M Primer
1.00 μL DN

After a 2 minute hot start at 94° C. the samples were run using the above conditions. The

PCR product was run on a 2% gel with a 100 bp ladder as reference.

After a 2 minute hot start at 94° C. the samples are run using the above conditions. The PCR product is run on a 2% gel with a 100 bp ladder as reference. The expanded ES clone, which is used as a positive control, is denoted by a (+) in the gel photograph in FIGS. 2-8.

Primer set NDEL1 and NDEL2 is used to screen mice for the deletion of the Neo cassette. The PCR product for the wild-type is 386 bp. After Neo deletion, one set of LoxP-FRT sites remain (159 bp). A second band with a size of 545 bp indicates Neo deletion. A single band of 386 bp indicates a homozygous mouse, two bands 386 and 545 bp in size indicates a heterozygous mouse, and a single band 545 bp in length indicates a wild type mouse.

### EXAMPLE 11

Breeding Cnr2 mice with various Cre recombinant mice.

CB2 flox mice are engineered and generated for the production of cell type selective deletion of CB2R receptors. The mutant, heterozygous and wild type CB2 flox mice are confirmed by genotyping and “without differences following behavioral characterization” using locomotor activity and emotionality tests. Breeding pairs are set up so as to continue a colony of the CB2 flox line. Simultaneously, DAT-Cre and Cx3crl-Cre mice are commercially obtained (Jackson Laboratories) and are crossed with the Cnr2-flox mice to generate DAT-Cnr2 and Cx3crl-Cnr2 lox transgenic mice and their wild type litter mates. The strategy is to keep these lines breeding. Selected breeding pairs are mated when they are sexually mature (6 to 8 weeks old).

Cnr2-flox mice are breed with Jackson Laboratory (1600 Maine Street, Bar Harbor, Me. 04609) DAT-Cre homozygous mice (B6-SJL-SLc6A3-CreJ) and generate DAT-Cnr2 transgenic mice for studying drug addiction and Parkinson's disease.

Crn2-flox mice are breed with Jackson Laboratory (1600 Maine Street, Bar Harbor, Me. 04609) Cx3crl-Cre mice (B6J.B6N(Cg)-Cx3crltml.1(cre)Jung/J) to generate Cx3crl-Cnr2 transgenic mice for studying inflammation associated diseases such as stroke, Alzheimer's disease and cancer.

Cnr2-flox mice are breed with the neural progenitor cells (NPC) specific gene, Nestin promoter linked Cre recombinase mice (B6.Cg-Tg(Nes-cre)1Kln/J) for studying stroke and Alzheimer's disease.

Cnr2-flox mice are breed with oligodendrite specific gene, Olig2 promoter linked Cre recombinase mice (B6.129-Olig2^{tml.1(cre)Wdr}/J) for studying autoimmune diseases such as multiple sclerosis. (Obtained from Jackson Laboratories)

The Cre gene selected for use in the present invention is selected from mouse strains of B6;SJL-Slc6a3^{tml.1(cre)Bkmn}/J; B6J.B6N(Cg)-Cx3cr1^{tml.1(cre)Jung}/J; B6Cq-Tg(Nes-Cre) 1Kln/J; or B6.129-Olig2^{tml.1(cre)Wdr}/J; sppl-Cre; Opn-CnR2; or IL6-Cre.

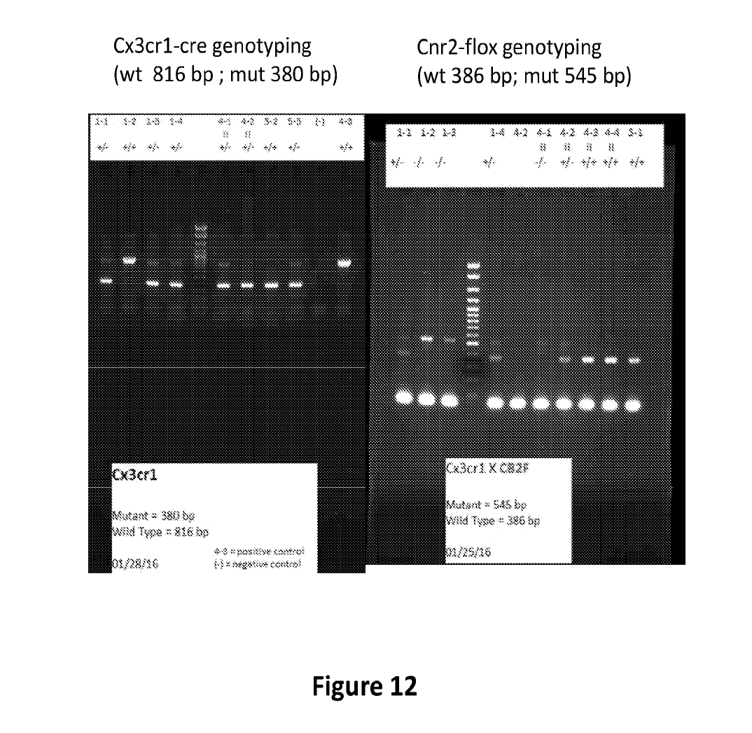

Cx3crl-Cnr2 microglia conditional knockout mice are created by crossing Cnr2-floxed mice with Cx3crl-Cre recombinase mice (Table 1 for primer sequences and FIG. 12 genotyping of Cx3crl-Cnr2 mice). Both the F1 and F2 generation of the Cx3crl-Cnr2 mice are obtained. The F1 generation of CB2F (includes eleven males and seven females). These then are used to obtain the F2 generation of Cx3Crl-Cnr2 lox mice, wherein five males and seven females are cross bred with ten male and ten females CB2F mice. Five mice with Cnr2 flox that are homozygous and heterozygous Cx3crl-Cre are found in FIG. 12. These mice are mated to produce Cnr2-flox and Cx3crl-Cre double allele homozygous mice for use in the mouse inflammation disease model.

Inbreed strains are produced by sibling matings, and in order to optimize the breeding performance two females are placed (one of them will be a proven breeder) and one male per cage. In some cases one male and one female are placed in the cage. To rapidly produce animals, two females are rotated through a male's cage every 1 or 2 weeks with nesting material placed in the cage, and animals receive breeder chow and water ad libitum. Litters are expected within a month of mating since female mice go into estrus every 3 or 4 days, and the gestation time of mice is 19-21 days. Males are removed from the cage right before or after the females give birth to prevent overcrowded cages or cannibalism. Ear tag or toe clipping are performed when pups are two weeks old. Tail biopsies for genotyping are obtained at the same time. The spread sheet is set up to keep track of breeding performance and track of the mice.

The weaning age depends on size and maturity of the pups, usually between 21 and 28 days old. If no litters are produced after one month, the animals are separated and replaced by new trios. Typically, mice breed for about 7 or 8 months. After that time period the breeders are replaced for younger animals. Other breeding records of mice inventory of animals indicates mouse ID, date of birth, parents, gender and genotype is shown in Table 5.

The scheme for the production of the F2 generation that is used for drug discovery and mechanistic studies is provided below, including the intermediate phenotypes for the Cnr2-flox and DAT-Cre or Cx3crl-Cre.

### EXAMPLE 12

Genotyping protocols of the DAT- Cre, Cx3crl-Cre and CB2 flox mice.

The protocols for isolating mouse tail DNA and performing DAT-Cre, Cx3crl-Cre and the Cnr2-flox mice genotyping by polymerase chain reaction (PCR) have similarities in set up. Gel electrophoreses with differences in the primers used for DAT-Cre, Cx3crl-Cre and the Cnr2-flox mice are described below with a prototype example. (Cre recombinant mice from Jackson Laboratories)

### Tail DNA Preparation

A 2-mm piece of tail tissue is cut and placed into a 0.5 mL PCR tube. 75 L alkaline lysis buffer (25 mL NaOH, 0.2 mM disodium EDTA, adjusted to a pH=12) is added to 0.5 mL tube. The disodium EDTA acts as a chelating agent. NaOH (strong base & pH=12) denatures DNA and proteins and degrades RNA. This is placed in a tube in the PCR machine and incubated at 95° C. for 30-60 minutes. The heated sample is placed on ice to cool for 5 min. 75 L of neutralizing buffer is added (40 mM Tris-HCl, pH 5.0) to each sample and then the samples are mixed.

### Master Mix (MM) Per Reaction is as Follows:

| | | |
|---|---|---|
| Taq Polymerase | 0.06 | μl |
| (Econo Taq) | | |
| dNTPs (10 mM) | 0.12 | μl |
| MgCl₂ (25 mM) | 0.96 | μl |
| 10x Buffer | 1.2 | μl |
| IMR6625 (1 mm) | 1.2 | μl |
| (1:10 dilution) | | |
| IMR6626 (1:10 dilution) | 1.2 | μl |
| (1 mm) | | |
| IMR8292 (1:10 dilution) | 1.2 | μl |
| H₂O | 4.06 | μl |

### PCR

The PCR machine is turned on and all reagents are put on ice. The master mix (MM) component amounts (shown above) are multiplied by (# of samples+1). The primers are diluted in a 1:10 dilution. Pipetted corrected amounts are placed into 1 Eppendorf tubes and labeled MM. The tube is shaken vigorously, pipette 10 μl of MM into each PCR tube. 2 μl of DNA are pipetted into the corresponding PCR tube (1 DNA sample/tube). The PCR tubes are covered and centrifuge for a few seconds. The tubes are placed in a PCR machine (Run program under→MAIN→O9V-DAT). Denaturation accrues at 94° C. Annealing occurs at 65° C. and takes place at extension: 72° C. The reaction is done when the PCR says “forever.” The PCR stays at 10° until cancelled. While the PCR is running, gels are prepared for electrophoresis.

### Gel Electrophoresis

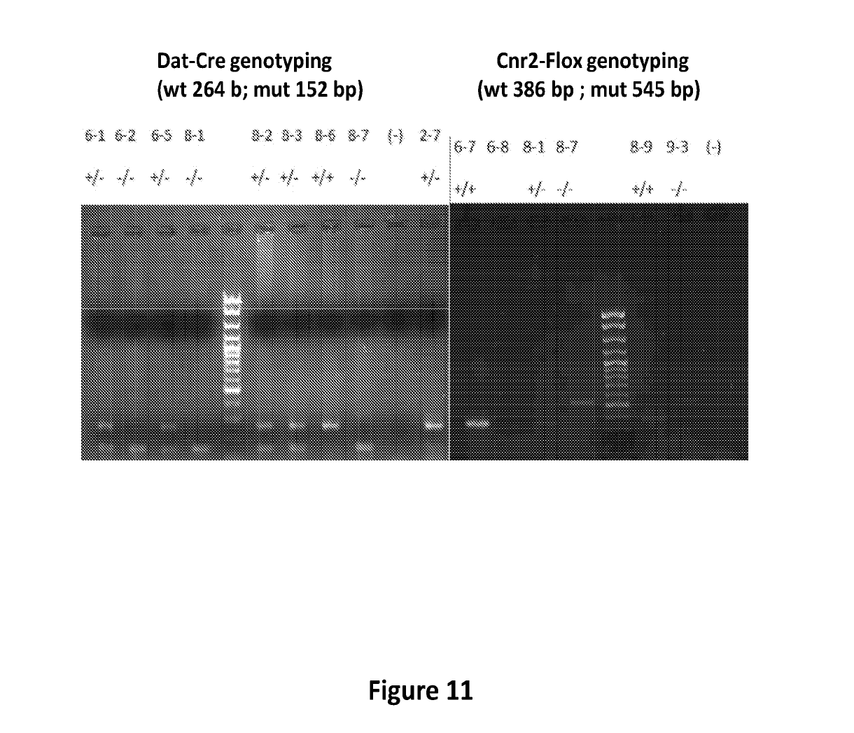

250 ml Erlenmeyer flask is used and 0.75 grams of agarose is added to it. 50 ml of TAE (a buffer solution containing a mixture of Tris base, acetic acid and EDTA) is added. The top of the flask is covered with Kim wipes, and then the flask is placed in a microwave for 30 seconds. The flask is then taken out, swirled, and placed back in the microwave for 20 seconds. This is repeated in 10 second intervals until all agarose is dissolved. 13.5 ul of ethidium bromide is added to flask and swirled. The sides of a gel container are taped and placed in the top and lower rows. The gels are poured into containers and let to solidify. After the PCR reaction is done there are 3 microliters of loading dye is added to each DNA sample. No EconoTaq Green Plus is needed. These are placed 6 ul of each sample with a ladder I nits respective well. The samples are covered and stored in a freezer in case in the event the gels need to be run again. The gel-electrophoreses cover with negative (black) terminal are placed at the top and positive (red) terminal at the bottom. DNA is slightly negative and will move towards the positive terminal. This runs for 50 minutes. Pictures are taken. Examples of PCR products for DAT-Cre, Cx3crl-Cre and the CB2 flox mice are provided in FIGS. 11 and 12.

### EXAMPLE 13

Protocols for Screening the Knockout Mice in Various Drug Delivery Assays.

Assessment of CB₂R mediated behavioral effects of DAT-Cnr2-Lox and WT mice is evaluated. Cannabinoid induced behavioral changes in the DAT-Cnr2-Lox and WT mice is used to determine the role of CB₂Rs in the mouse tetrad tests. Briefly, the mouse tetrad consists of four simple evaluations, which may be measured in sequence. They are as follows: Ten mins in the locomotor activity boxes, b). Catalepsy test, amount of time in 5 mins if the animal remains immobile, c). Rectal temperature and d). Nociception by the tail flick response.

The role of CB₂Rs in these cannabinoid induced effects determines conditional mutant mice when challenged with a specific agonist, JWH133 agonist or antagonist AM630 (N=10 animals per group, because of variability in behavioral studies) is studied. The data from this work sheds further light in the understanding that functional CB2Rs are present and expressed in dopamine neurons, and potential CB2R agonist as therapeutic agents in treating drug abuse and Parkinson's disease associated with dopamine neuron dysfunction.

### EXAMPLE 14

Optimal treatment time with CB2R agonist for anti-inflammation and neurodegeneration is studied after stroke. Early and pretreatment with CB2R selective agonists, synthetic cannabinoid JWH133 or AM1241 Tocris Bioscience (The Watkins Bldg. Atlantic Road, Avonmouth, Bristol, BS11 9QD, United Kingdom), significantly reduce brain infarct volumes and neurological deficits. Both CB2Rs mRNA and proteins are increased significantly in microglia and neurons after stroke in a time-dependent manner. Using Cx3crl-Cnr2 and Nestin-Cnr2 pre-clinical mouse models of stroke timed experiments by administering the commercially available CB2R agonist JW133 at specific time points post infarct at selected doses provides data to evaluate compounds for this use. The CB2R molecular pathways and partners in stroke studied in microglia and neural progenitor cells (NPC) on different post stroke days are evaluated when various compounds are tested. Such microglia and neuron specific CB2Rs-KO stroke behavioral models allow precise mapping of CB2R selective agonist (e.g. JWH133) for potential protective roles in stroke.

### EXAMPLE 15

CB2R agonists are identified to reduce hyperalgesia in multiple sclerosis. An experimental autoimmune encephalomyelitis, an animal model of the human CNS demyelinating diseases that involves t-cell mediated autoimmune disease, is used in olig2-Cnr2 oligodendrite cells specific to Cb2 conditional knockout mice. This is used to screen CB2R agonists as potential therapeutic agents for the treatment of central pain in an animal model of multiple sclerosis using somatosensory pain behavioral testing.

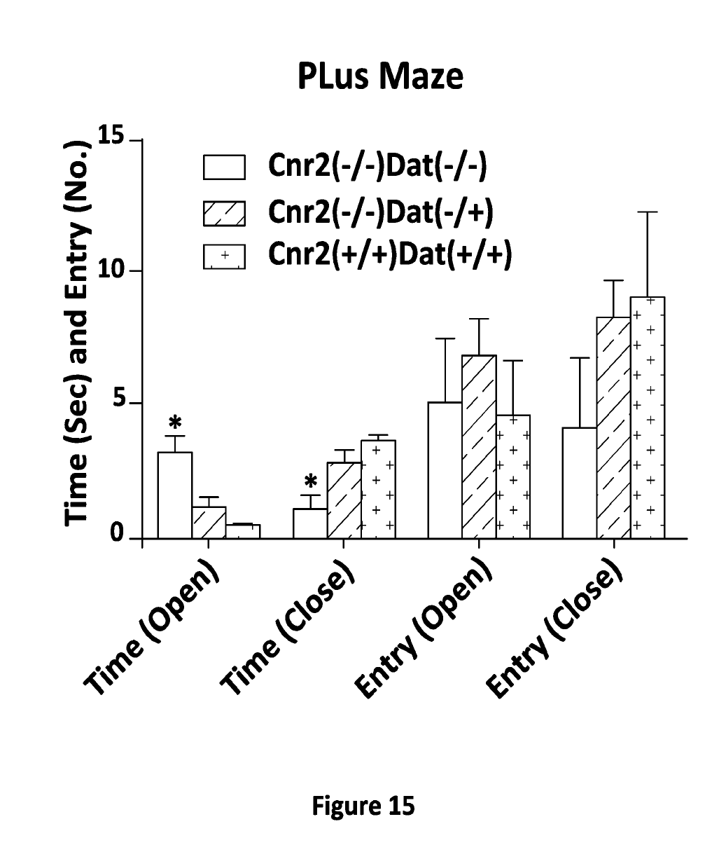

The performance of the DAT-Cnr2 in the plus-maze test of anxiety behavior, is evaluated and its found that Dat-Cnr2 homozygous mice are less anxious than heterozygous and wild type mice. (See FIG. 15)

### EXAMPLE 16

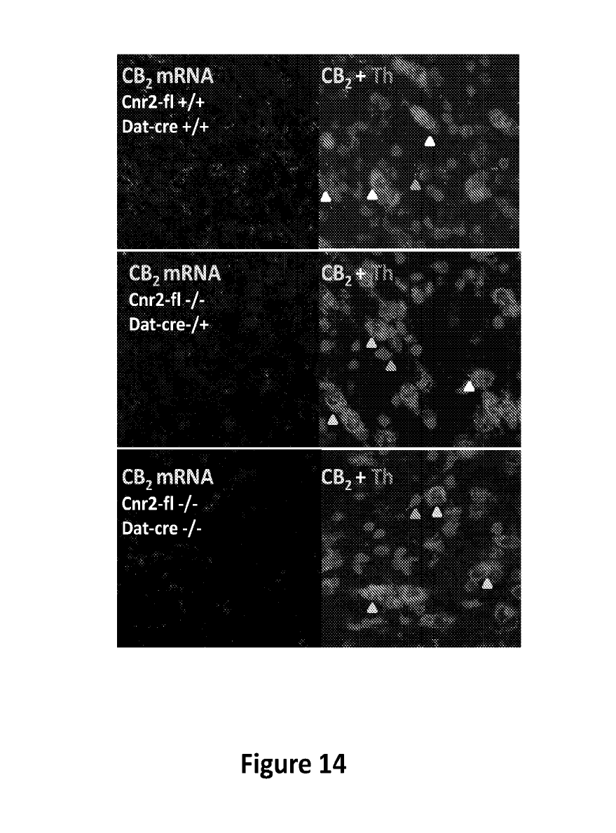

Dat-Cnr2 dopamine neuron conditional knockout mice are produced by crossing Cnr2-floxed mice with dopamine transporter promoter driven DAT-Cre recombinase mice and genotype of double allele homozygous mice are confirmed (Table 8, FIG. 11). The absence of CR2R mRNA in dopamine neurons is demonstrated by RNAscope in situ hybridization of mid brain ventral tegmental area (VTA) of wild type, heterozygous, and homozygous mice. (See FIG. 14, red/green Cnr2-flox−/−, Dat-Cre−/−; black/yellow Cnr1-flox−/−, Dat-Cre−/+)

TABLE 5
Inventory of the F2 generation Dat-Cnr2
dopamine neuron conditional knockout mice

| Dat-Cnr2 F2 | Cnr2-flox | Dat-Cre | Sex |
|---|---|---|---|
| 1-2 | −/− | +/− | Male (Yellow) |
| 1-5 | −/− | −/− | Male (Green) |
| 2-5 | −/− | −/− | Male (Green) |
| 4-1 | −/− | +/− | Male (Yellow) |
| 4-2 | −/− | +/− | Female (Yellow) |
| 4-3 | −/− | +/− | Female (Yellow) |
| 4-5 | −/− | +/− | Male (Yellow) |
| 5-4 | −/− | +/− | Male (Yellow) |
| 5-5 | −/− | −/− | Male (Green) |
| 5-6 | −/− | +/− | Female (Yellow) |
| 5-9 | −/− | +/− | Female (Yellow) |
| 6-1 | −/− | +/− | Female (Yellow) |
| 6-2 | −/− | −/− | Female (Green) |
| 6-5 | −/− | +/− | Male (Yellow) |
| 8-2 | −/− | +/− | Male (Yellow) |
| 8-3 | −/− | +/− | Male (Yellow) |
| 8-7 | −/− | −/− | Male (Green) |
| 9-3 | −/− | −/− | Male (Green) |

Genotyped F2 generation are developed and identified as Dat-Cnr2 dopamine neuron conditional knockout mice, e.g. #8-7 mouse (FIG. 12) that is homozygous in both Cnr2-flox and Dat-Cre alleles.

Provided below are the primers that are used in genotyping the Cnr2-flox mice (see Table 6). (Primer obtained from iTL). Also, the primers that are used for genotyping the DAT-cre mice are found in Table 7.

TABLE 6
The primers used for the Cnr2-flox mouse
genotyping:

| Primer | Sequence 5′→3′ | Primer Type |
|---|---|---|
| NDEL1 | GGT CAA GAA TTA TGA TGC CCT | Common |
| | AAG GAC | Forward |
| NDEL2 | CCC AAC TCC TTC TGC TTA TCC | Common |
| | TTC AGG | Reverse |

TABLE 7
The primers used for the DAT-cre mouse genotyping:

| Primer | Sequence 5′→3′' | Primer Type |
|---|---|---|
| IMR6625 | TGG CTG TTG GTG TAA | Common Forward |
| | AGT GG | |
| IMR6626 | GGA CAG GGA CAT GGT | Wild type Reverse |
| | TGA CT | |
| IMR8292 | CCA AAA GAC GGC AAT | Mutant Reverse |
| | ATG GT | |

TABLE 8
The primers used for the Cx3cr1-cre mouse
genotyping are as follows:

| Primer | Sequence 5′→3′ | Primer Type |
|---|---|---|
| 20669 | AGC TCA CGA CTG CCT | Wild type Forward |
| | TCT TC | |
| 20670 | GCA GGG AAA TCT GAT | Common |
| | GCA AG | |
| 21250 | GAC ATT TGC CTT GCT | Mutant Forward |
| | GGA C | |

20669, 206702 and 21250 are labels for Cx3crl-Cre mice genotyping.

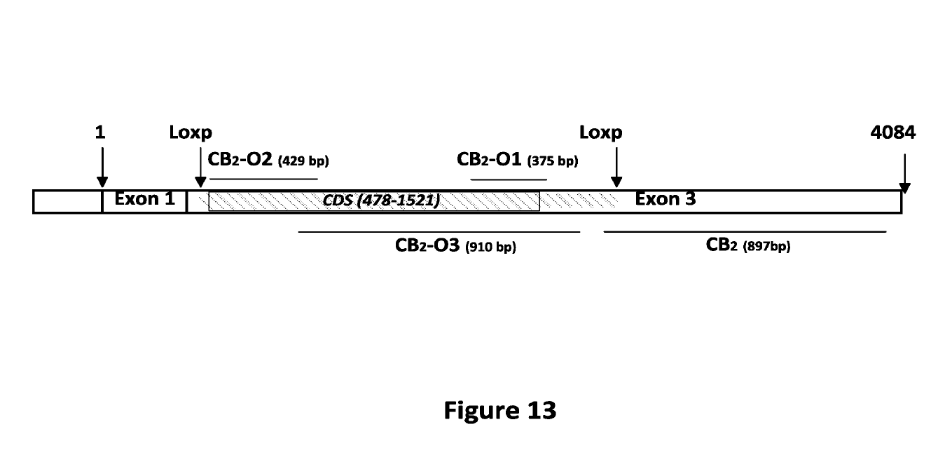

Additionally, the deletion of Cnr2 mRNA in the midbrain vental tegmental area (VTA) dopamine neurons by RNAscope in situ hybridization (ISH) using probe (see FIG. 13 probe positions) is a verification that confirms targeting to Cnr2-floxed region (FIG. 14, probe positions, CB2 mRNA deleted in DA neurons).

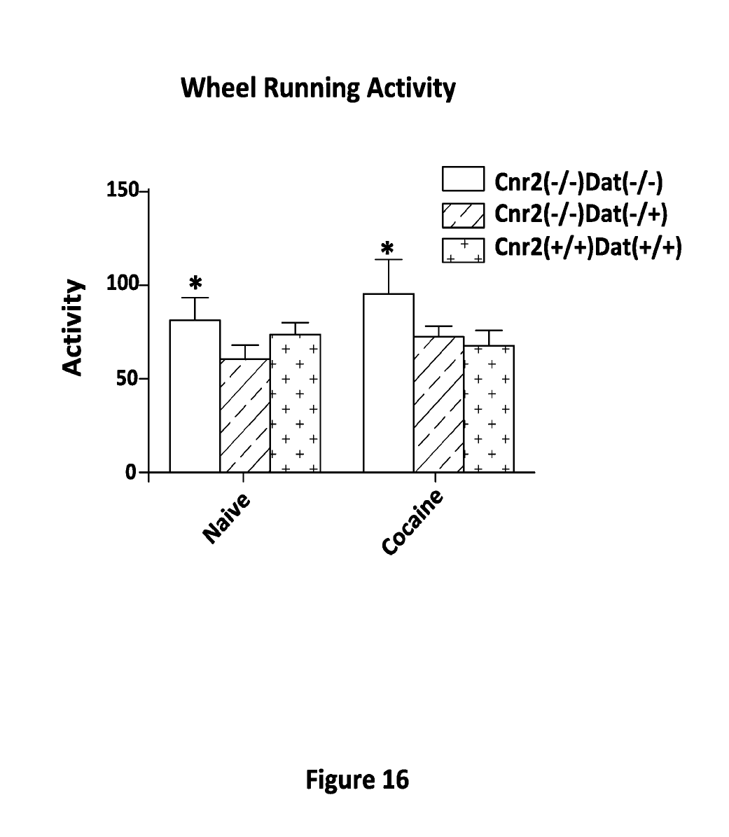

The anxiety test is evaluated by elevated plus-maize behavioral measurement. The longer time of mice staying in the open arm represents less anxious mice so do the less time of mice staying in the close arm. Dat-Cnr2 mice are statistically less anxious than wild type and heterozygous mice (FIG. 15). The motor function test is evaluated by observing the effects of cocaine (a psychostimulant) in the Dat-Cnr2 mice by measuring wheel running activity. It is found that Dat-Cnr2 homozygous mice are more responsive to cocaine stimulation of motor activity than heterozygous and wild type mice (FIG. 16, n=4-6).

### EXAMPLE 17

Utilizing the procedures outlined above, the following Table 9 provides the specifics of the transgenic mice tested and useful in mouse models to test for effects of compounds for treating damaged neurons, dopaminergic neurons such as found in Parkinson's disease, stroke and multiple sclerosis. Furthermore, these mouse models are effectively used in screens for drug abuse.

TABLE 9

| | | | Disease state | Known drug |
|---|---|---|---|---|
| | | Resulting KO mic [symbol] | Being | screening assay |
| LoxP Mous [symbol] | Cre Mouse | gene removed | researched | and biomarker |
| Cnr2-flox | B6-SJL-Slc6A3-CreJ | DA neuron | Drug abuse | JWH 133 |
| | Cx3cr1tm1.1(cre)Jung/J | Microglia | & Parkinson | GP1a |
| | B6.Cg-Tg(Nes-cre) | Neuroprogenitor | Stroke | GW405833 |
| | 1Kln/J | Oligodendrite | Multiple- | HU308 |
| | B6.129- | | Sclerosis | |
| | Olig2^{tm1.1(cre)Wdr}/J | | | |

[symbol]

indicates data missing or illegible when filed

The behavioral effects of CB2R activation and its influence on food and alcohol consumption in mice have been evaluated using the ko mice. CB₂Rs in the brain play a role in food and alcohol consumption, and data demonstrate a role of central CB₂Rs on food intake in neonatal chicks. (1. Alizadeh A, Zendehdel M, Babapour V, Charkhkar S, Hassanpour S, Role of cannabinoidergic system on food intake in neonatal layer-type chicken. Veterinary research communications. 2015; 39(2):15-7. doi: 10.1007/s11259-015-9636-3. PubMed PMID: 25902906. 2. Ortega-Alvaro A, Ternianov A, Aracil-Fernandez A, Navarrete F, Garcia-Gutierrez M S, Manzanares J. Role of cannabinoid CB2 receptor in the reinforcing actions of ethanol. Addiction biology. 2015; 20(1):43-55. doi: 10.1111/adb.12076. PubMed PMID: 23855434) (all of which are incorporated herein by reference).

### EXAMPLE 18

CB2R agonists are useful as potential therapeutic agents for treating osteoporosis. CB2-deficient mice show a markedly accelerated age-related bone loss and the CNR2 gene (encoding CB2R) in women is associated with low bone mineral density after menopause (Bab I, Zimmer A. Cannabinoid receptors and the regulation of bone mass. British journal of pharmacology. 2008; 153(2):182-8. doi); 10.1038/sj.bjp.0707593. PubMed PMID: 18071301; PubMed Central PMCID: PMC2219540 (all incorporated by reference). Activation of CB2R enhances osteogenic differentiation of bone marrow mesenchymal stem cells (Sun Y X, Xu A H, Yang Y, Zhang J X, Yu A W. Activation of cannabinoid receptor 2 enhances osteogenic differentiation of bone marrow derived mesenchymal stem cells. BioMed research international. 2015; 2015:874982. doi (all incorporated by reference); 10.1155/2015/874982. PubMed PMID: 25685815; PubMed Central PMCID: PMC4317596. Opn-Cnr2 mice with osteocyte specific deletions of Cnr2 as a osteoporosis animal model, are provided in the present invention (Opn)-Cre mice.

CB2R agonist Ajulemic acid (Resunab™) is in the accelerated FDA approval process for the treatment of Systemic sclerosis—scleroderma (Gonzalez E G, Selvi E, Balistreri E, Akhmetshina A, Palumbo K, Lorenzini S, Lazzerini P E, Montilli C, Capecchi P L, Lucattelli M, Baldi C, Gianchecchi E, Galeazzi M, Pasini F L, Distler J H. Synthetic cannabinoid ajulemic acid exerts potent antifibrotic effects in experimental models of systemic sclerosis. Annals of the rheumatic diseases. 2012; 71(9):1545-51. doi: 10.1136/annrheumdis-2011-200314. PubMed PMID: 22492781) (incorporated by reference), an autoimmune disease characterized by abnormalities in blood vessels and thickening of the skin caused by pathological accumulation of collagen. IL6-Cnr2 mice with macrophage specific deletion of Cnr2 as an animal model is provided to study effectiveness. (IL6)-Cre mice are available from Jackson Laboratories.

### EXAMPLE 19

As can be seen from the presently provided description, two types of transgenic mice are generated. One type is a Cnr2-flox with Neo for producing Cnr2-flox mice without Neo. Further, cell type specific deletions of Cnr2 are derived by mating conditional knockout mice of Cnr2-flox mice and Cre expressing mice or other appropriate mouse models. In particular, dopaminergic neuron, microglia, neural progenitor and oligodendrite cell types as well as osteocyte specific deletions of Cnr2 are provided. A Cnr2-Cre mouse is also available. Other Cnr2 combinations are also available and are ones those of ordinary skill in the art recognize as part of the present invention.

1. An animal model comprising a floxed Cnr2 gene.

2. The animal model according to claim 1 further comprising the Cnr2 gene and a Neo gene.

3. The animal model according to claim 2, wherein the Neo gene is deleted; and is flanked with LoxP.

4. The animal model according to claim 3, wherein the animal is a mouse.

5. The animal model according to claim 4, wherein SEQ ID NO: 1 is the gene sequence of the Cnr2 coding sequence.

6. The animal model according to claim 2, wherein the animal is a mouse.

7. The animal model according to claim 3, wherein the Cre gene is selected from mouse strains of B6; SJL-Slc6a3^{tml.1(cre)Bkmn}/J; B6J.B6N(Cg)-Cx3crl^{tml.1(cre)Jung}/J; B6Cq-Tg(Nes-Cre) 1Kln/J; or B6.129-Olig2^{tml.1(cre)Wdr}/J; sppl-Cre; Opn-CnR2; or IL6-Cre.

8. The animal model according to claim 7, wherein the Cre gene is B6-SJh-Slc6A3-CreJ.

9. The animal model according to claim 7, wherein the Cre gene is B6J.B6N(Cg)Cx3cr/tml.1(Cre)Jung/J.

10. The animal model according to claim 7, wherein the Cre gene is under tissue specific promoter control of mouse genes of Slc6A3, Cx3crl, Nestin, Olig2, Osteopontin, and Interleukin-6, respectively.

11. The animal model according to claim 7, wherein the Cre gene is B6.129-Olg₂.

12. The animal model wherein the Cre gene is (LL6)-Cre.

13. The animal model wherein the Cre gene is Opn.

14. The animal model according to claim 7 wherein said gene is removed from the group selected from dopamine neurose, microglia, macrophage, osteoblast, neuroprogentor cells or oligodendritic cells, osteoporosis.

15. The animal model according to claim 7, wherein said animal model is used to screen for drug abuse, Parkinson's Disease, Post-Stroke Inflammation, Multiple Sclerosis, drug addiction, alcoholism, inflammation, osteoporosis, autoimmune disease, scleroderma and/or cancer.

16. A method for selecting a drug that targets CB₂R, said method comprising:
(a) administering a drug to a mouse model wherein said model has the Cnr2 gene;
(b) measuring the binding activity of a drug in the mouse model of wild type and cell type specific Cnr2 mice;
(c) selecting the drug to provide to a patient;
(d) administering the selected drug to a patient in need thereof.

17. The method according to claim 16, wherein said mouse model additionally comprises a Cre gene.

18. The method according to claim 16, wherein the in vitro and/or in vivo activity of a drug to cell type specified CB₂ receptor activity is determined.

19. The method according to claim 18, wherein said mouse model comprises the Cnr2 gene and a Cre gene selected from the group 6-SJL-Slc6A3-creJ, B6J.B6N(Cg)Cx3 critm6.1(cre) Jug/J, B6Cq-Tg(Nes-Cre) 1Kn_{d}; or B6.129-Olig₂, Opn-Cre, or (IL6)-Cre.

20. The method according to claim 19, wherein the activity is activation.

21. The method according to claim 19, wherein the activity is inhibition.

22. A method for producing a conditional CB2R knockout mouse, said method comprising :
(a) mating a Cre mouse with a Lox P mouse resulting in a Cnr2 gene deletion mouse;
(b) taking the resultant Cnr2 gene deletion mouse;
(c) incorporating the Cnrᵣ deleted gene; and
(d) administering a drug to said resultant mouse to measure the activation or inhibition of the gene.

23. The method according to claim 22, wherein the Cre mouse is selected from the group B6-Sjh-Slc6A3-creJ, B6J.B6N(Cg)Cx3 critm6.1(cre) Jug/J, B6Cq-Tg(Nes-Cre) 1Kn_{d}; or B6.129-Olig₂, Opn-Cre, or (IL6)-Cre.

24. The method according to claim 23, wherein the Cre mouse is B6-Sjh-Slc6A3-CreJ.

25. The method according to claim 23, wherein the Cre mouse is B6J.B6N(Cq)Cx3 critn6.1(Cre).

26. The method according to claim 23, wherein the Cre mouse is Jug/J,B6(q-Tg(Nes-Cre)1 knd.

27. The method according to claim 23, wherein the mouse is B6.129-Oliq₂.

28. The method according to claim 23 wherein the resultant mouse is used for testing drugs to treat or prevent psychosis, anxiety, depression, autism disorder, drug addiction, Parkinson's disease, Alzheimer's disease, Multiple Sclerosis, Post-Stroke inflammation, osteroporasis, autoimmune disease, scleroderma or cancer.

29. A floxed Cnr2 gene.

30. The floxed Cnr2 gene according to claim 29, wherein said gene is SEQ ID NO: 1.

31. A cassette comprising a floxed Cnr2 gene.

32. The cassette according to claim 31, wherein said gene is in SEQ ID NO: 1.

33. The construction according to claim 32 comprising:
(a) an open reading frame of Example 3;
(b) a splicing site of the open reading frame;
(c) A Neo cassette for antibiotic marker; and
(d) a 5′ acceptor, Exon 3 receptor splicing site;
wherein the Cn2-floxed gene is flanked by loxP sequences.

34. A transgenic mouse comprising a floxed Cnr2 gene.

35. The mouse according to claim 34, wherein the Neo gene is deleted and is flanked with LoxP.

36. The mouse according to claim 35 comprising:
(a) a Cre gene; and
(b) LoxP flanking the Cnr2 coding region.

37. The mouse according to claim 35, wherein the Cre gene is selected from B6-Sjh-Slc6A3-creJ, B6J.B6N(Cg)Cx3 critm6.1(cre) Jug/J, B6Cq-Tg(Nes-Cre) 1Kn_{d}; or B6.129-Olig₂, Opn-Cre; or (IL6)-Cre.

38. The mouse according to claim 37, wherein the Cre gene is B6-SJh-Slc6A3-CreJ.

39. The mouse according to claim 37, wherein the Cre gene is B8J.B6N(Cq)Cx3 critm 6.1 (cre).

40. The mouse according to claim 37, wherein the Cre gene is B6(q-Tq(Nes0Cre) 1Kn₂.

41. The mouse according to claim 37, wherein the Cre gene is B6.129-Olg₂.

42. The mouse according to claim 37, wherein the Cre gene is Opn-Cre.

43. The mouse according to claim 37, wherein the Cre gene is (IL6)-Cre.

44. A method for testing a drug that targets CB₂R, said method comprising:
(a) administering a drug to a mouse model where in said model has the Cnr2 gene;
(b) measuring the binding activity of a drug to the CB₂R mouse model;
(c) selecting the drug to provide to a patient;
(d) administering the selected drug to a patient in need thereof.

45. The method according the claim 39, wherein the Cnr2 gene sequence is in SEQ ID NO: 1.
